(19) United States
(12) Patent Application Publication
(10) Pub. No.: US 2004/0002479 A1
(43) Pub. Date: Jan. 1, 2004

(54) Peptide analogues and uses thereof

(76) Inventors: Yihan Wang, Newton, MA (US); Tomi K. Sawyer, Southborough, MA (US); David L. Berstein, Waban, MA (US)

Correspondence Address: David L. Berstein, ARIAD Pharmaceuticals, Inc., 26 Landsdowne Street, Cambridge, MA, 02139-4234, US

(21) Appl. No.: 10/386,649

(22) Filed: Mar. 12, 2003

Related U.S. Application Data
(60) Provisional application No. 60/363,961, filed on Mar. 12, 2002.

(51) Int. Cl. A61K 31/675; A61K 31/66; C07F 9/547; C07F 9/28

(52) U.S. Cl. 514/79; 514/89; 514/114; 546/22; 548/413; 558/168

(57) ABSTRACT

This invention relates to compounds of the general formula:

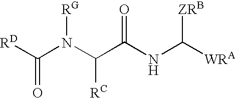

in which R^{A}, R^{B}, R^{C}, R^{D}, R^{G} and Z are as defined herein, and to their preparation and use.

### BACKGROUND OF THE INVENTION

Because of the wide range of cellular substrates and processes controlled by the ubiquitin-proteasome pathway, proteasome inhibitors have attracted recent interest for treating a variety of diseases including, among others, various cancers, inflammation, sepsis and AIDS. For example, the oscillation of cyclins (cell cycle proteins required for the orderly progression through the cell cycle) has been attributed to the regulated protein degradation mediated by the ubiquitin-proteasome pathway, and inhibition of this pathway is believed to result in the blockage of cell cycle progression. Additionally, the action of transcriptional factor NF-kB, another regulatory protein involved in a variety of cellular processes including immune and inflammatory responses, apoptosis, and cellular proliferation, is controlled by the ubiquitin-proteasome pathway. Furthermore, it has also been shown that the ubiquitin-proteasome pathway is involved in retrovirus assembly and thus may be useful for the development of anti-HIV drugs. For a general discussion of the ubiquitin-proteasome pathway and proteasome inhibitors see, Myung et al. “The Ubiquitin-Proteasome Pathway and Proteasome Inhibitors” Medicinal Research Reviews 2001, 21, 245-273. See also U.S. Pat. Nos. 6,465,433; 6,297,217 and 6,083,903; WO 02/059131; and Kisselev and Goldberg, Chemistry & Bioology (2001) 739-758; and references cited therein.

Clearly, it would be useful to develop novel therapeutics capable of inhibiting proteasome activity and exhibiting a desirable therapeutic effect. More generally, however, there remains a need for the development of novel therapeutics, regardless of their mechanism of action, for the treatment of debilitating disorders such as any of the various cancers, sepsis, AIDS, osteoporosis and other diseases involving untoward bone resorption (e.g., Paget's Disease, primary and secondary hyperparathyroidism, humoral hypercalcemia of malignancy, various cancers where resorption is increased, and rheumatoid arthritis), and inflammatory disorders, to name a few. New agents with attractive physicochemical or functional characteristics, e.g., with respect to therapeutic index, bioavailability, pharmacokinetics, stability, etc., would be of interest for a variety of pharmaceutical uses such as are mentioned above.

1. General Description of Compounds of the Invention:

This invention provides a novel family of peptide analogues having useful biological and pharmacological properties.

These include compounds having the general formula (I):

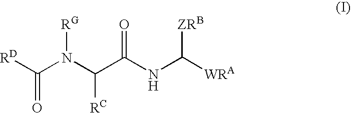

and pharmaceutically acceptable derivatives thereof,

wherein R^{A} and R^{B} are each independently hydrogen, COOH, B(OH)₂, a phosphorus-containing moiety, or an ester of any of the foregoing, or an aliphatic, heteroaliphatic, aryl or heteroaryl moiety;

Z and W are each independently a covalent bond or an aliphatic, heteroaliphatic, aryl, or heteroaryl linker of 1-10 carbon atoms;

R^{C} is an aliphatic, heteroaliphatic, aryl, or heteroaryl moiety (including among others aryl or heteroaryl moieties bearing one or more aliphatic or heteroaliphatic substituents, e.g., alkylaryl-, or alkylheteroaryl-moieties);

R^{D} is an aryl or heteroaryl moiety;

R^{G} is hydrogen or an aliphatic group;

wherein in each of the foregoing groups Z, W, R^{C}, R^{D}, and R^{G} each alkyl, aliphatic, heteroaliphatic, moiety may independently be branched or unbranched, cyclic or acyclic or substituted or unsubstituted, and may contain one or more electronically unsaturated bonds, and each aryl and heteroaryl moiety may independently be substituted or unsubstituted; and

at least one of R^{A}, R^{B}, R^{C} or R^{D} comprises or is substituted with a phosphorus-containing moiety. Illustrative phosphorus-containing moieties include the following (where the variable groups are as defined below, e.g., in connection with Series I):

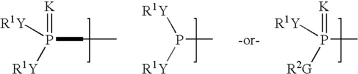

In certain embodiments of the invention,

(i) if R^{A} is the only phosphorus-containing moiety; if R^{D} is a phenyl moiety substituted with a substituted or unsubstituted phenoxy or naphthyloxy moiety; and if -ZR^{B} is a phenyl, benzyl or C₁₋₆ alkyl moiety substituted with an amidino, guanidino, isothioureido or amino moiety, then R^{A} is not —P(═O)(OR^{Z})(R^{X}), where R^{Z} is C₁₋₆ perfluoroalkyl, phenyl or substituted phenyl and R^{X} is C₁₋₆ perfluoroalkoxy, phenoxy, halogen, C₁₋₆ alkoxy or substituted phenoxy;

(ii) if R^{A} is the only phosphorus-containing moiety; if ZR^{B} is an alkyl, alkenyl, or alkynyl moiety, wherein any hydrogen bound to a carbon atom is optionally substituted with halogen, and wherein any hydrogen or halogen atom bound to any terminal carbon atom is optionally substituted with sulfhydryl or hydroxy; if R^{C} is an alkyl, cycloalkyl, aryl, aralkyl (also referred to as “arylalkyl”, i.e., aryl-substituted alkyl, such as benzyl or substituted benzyl), heterocycle, heterocyclylalkyl, heteroaryl, or heteroaralkyl, optionally substituted by 1-3 of alkyl, aryl, aralkyl, alkoxy, aryloxy, aralkoxy, cycloalkyl, cycloalkoxy, heterocycle, heterocyclyloxy, keto, hydroxy, amino, alkylamino, alkanoylamino, aroylamino, aralkanoylamino, carboxy, carboxyalkyl, carboxamidoalkyl, halo, cyano, nitro, formyl, acyl, sulfonyl or sulfonamido, optionally further substituted with 1-3 of alkyl, aryl, aralkyl, alkoxy, aryloxy, heterocycle, heterocyclyloxy, keto, hydroxy, amino, alkanoylamino, aroylamino, carboxy, carboxyalkyl, carboxamidoalkyl, halo, cyano, nitro, formyl, sulfonyl, or sulfonamido; and if R^{D} is a substituted or unsubstituted aryl or heteroaryl moiety, then R^{A} is not —P(═O)((O)_{Y}R^{Y})((O)_{Y}R^{Y}) or —P((O)_{Y}R^{Y})((O)_{Y}R^{Y}), where Y is 0 or 1 and each R^{Y} is independently hydrogen, alkyl, alkenyl, aryl, aralkyl, aralkenyl, cycloalkyl, cycloalkylalkyl, cycloalkenyl, cycloalkenylalkyl, heterocycle, heterocyclylalkyl, heterocyclyalkenyl, heteroaryl or heteraralkyl; or

(iii) if R^{D} is the only phosphorus-containing moiety and is a quinoline moiety substituted with —PO₃H; then R^{C} is not an alkyl, aryl, or aralkyl moiety substituted with nitro or amino.

Illustrative, non-limiting examples of phosphorus-containing moieties are depicted by the moieties shown in Series I, Ia, and Ib discussed below:

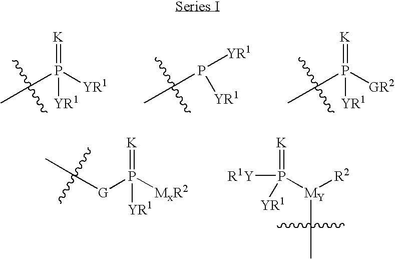

wherein each occurrence of K is independently O or S;

each occurrence of Y is independently —O—, —S—, —NR¹—, or a chemical bond linking R¹ to P,

each occurrence of R¹ is independently a substituted or unsubstituted aliphatic, heteroaliphatic, aryl or heteroaryl moiety, or, except in YR¹ moieties in which Y is a covalent bond, R¹ may also be H;

each occurrence of R² is independently R¹, —PK(YR¹)(YR¹), —SO₂(YR¹) or —C(O)(YR¹);

each occurrence of G is independently absent, or is —O—, —S—, —NR¹— or (M)_{X};

each occurrence of M is independently a substituted or unsubstituted methylene moiety, and any M-M′ moiety may be saturated or unsaturated;

each occurrence of x is independently an integer from 0-6; and

each occurrence of M_{Y} is independently a methine group or a lower alkyl moiety which contains a methine group and optionally may be further substituted.

Phosphorus-containing moieties are further illustrated by the groups of Series Ia and Ib below:

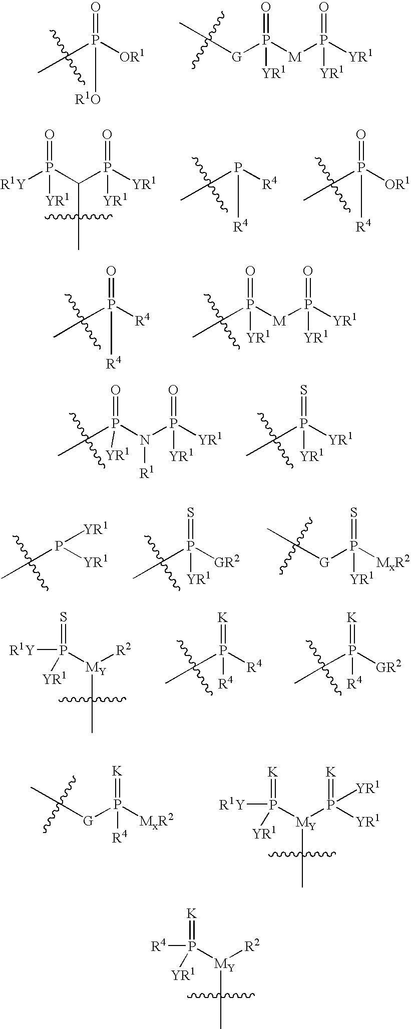

wherein each occurrence of R⁴ is independently an aliphatic, heteroaliphatic, aryl, or heteroaryl moiety, and other variable groups are as defined for Series I above.

Note that in the various embodiments two R groups in the phosphorus-containing moiety may be linked together to form a ring as illustrated by the following phosphorus-containing moieties:

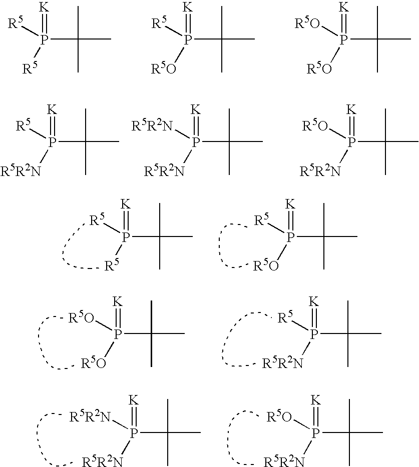

and exemplified by the following:

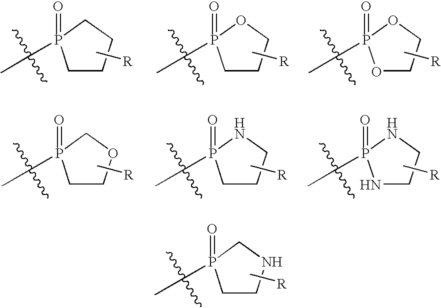

and homologous moieties containing 6-membered or larger rings and/or rings containing double bonds.

2. Featured Classes of Compounds of the Invention:

One class of compounds of special interest includes those compounds in which R^{C} is a benzyl moiety optionally substituted with one or more occurrences of R³ and R^{E} and the compound has the structure (2):

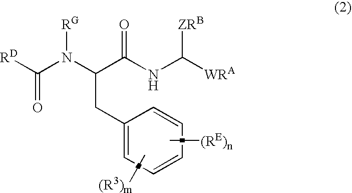

wherein each occurrence of R³ is independently halogen; -GR¹; -GCO(YR¹); -GSO₂(YR¹); cyano, nitro or azido (e.g., including among others, hydrogen; halogen; R¹; —OR¹; —SR¹; —NR¹R^{1′}; —CO(YR¹); —CO(NHR¹); —NHCO(YR¹); cyano, nitro, azido, sulfonate, sulfonamido, etc.);

each occurrence of Y, R¹, G, M and x are as previously defined;

one or more of R^{A}, R^{B}, R^{D} or R^{R} comprises or is substituted with a phosphorus-containing moiety;

n is 0-3; and

m is 0-3, and the sum of n+m is an integer from 0-5.

In certain embodiments of the compounds described directly above, if R^{D} is the only phosphorus-containing moiety and is a quinoline moiety substituted with the phosphorus-containing moiety; then R^{C} is not an alkyl, aryl, or aralkyl moiety substituted with nitro or amino.

In certain other embodiments of the compounds described directly above, if R^{A} is the only phosphorus-containing moiety; if R^{D} is a phenyl moiety substituted with a substituted or unsubstituted phenoxy or naphthyloxy moiety; and if —ZR^{B} is a phenyl, benzyl or C₁₋₆ alkyl moiety substituted with an amidino, guanidino, isothioureido or amino moiety, then R^{A} is not —P(═O)(OR^{Z})(R^{X}), where R^{Z} is C₁₋₆ perfluoroalkyl, phenyl or substituted phenyl and R^{X} is C₁₋₆ perfluoroalkoxy, phenoxy, halogen, C₁₋₆ alkoxy or substituted phenoxy.

Another class of special interest includes those compounds wherein ZR^{B} is —CH₂CH(CH₃)(CH₃) and the compound has the structure (3):

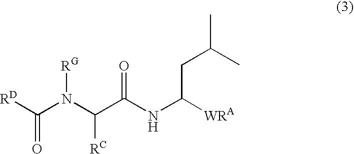

wherein R^{A}, R^{C}, R^{D}, R^{G} and W are as defined generally above as defined in any of the classes and subclasses described elsewhere herein and wherein at least one of R^{A}, R^{C} or R^{D} comprises or is substituted with a phosphorus-containing moiety.

In certain embodiments of the compounds described directly above, if R^{D} is the only phosphorus-containing moiety and is a quinoline moiety substituted with —PO₃H; then R^{C} is not an alkyl, aryl, or aralkyl moiety substituted with nitro or amino.

Another class of compounds of special interest includes those compounds wherein R^{D} is an aryl moiety optionally substituted with one or more of moieties R^{F} and R³, and the compound has the structure (4):

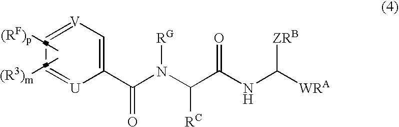

wherein each occurrence of R³, Y, R¹, G, M and x is as defined previously or in the various classes and subclasses herein;

at least one of R^{A}, R^{B}, R^{C} or R^{F} comprises or is substituted with a phosphorus-containing moiety;

V and U are each independently N or CR³;

p is 0-3; m is 0-3; and the sum of m+p is an integer from 0-5.

In certain embodiments of the compounds described directly above, if R^{A} is the only phosphorus-containing moiety; if R^{D} is a phenyl moiety substituted with a substituted or unsubstituted phenoxy or naphthyloxy moiety; and if -ZR^{B} is a phenyl, benzyl or C₁₋₆ alkyl moiety substituted with an amidino, guanidino, isothioureido or amino moiety, then R^{A} is not —P(═O)(OR^{Z})(R^{X}), where R^{Z} is C₁₋₆ perfluoroalkyl, phenyl or substituted phenyl and R^{X} is C₁₋₆ perfluoroalkoxy, phenoxy, halogen, C₁₋₆ alkoxy or substituted phenoxy.

Another class of special interest includes those compounds wherein WR^{A} is B(OH)₂ and the compound has the structure (5):

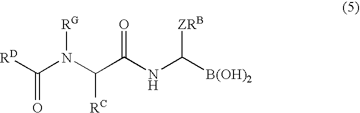

where Z, R^{B}, R^{C}, R^{D} and R^{G} are as defined generically above or in the various classes and subclasses herein, and wherein at least one of R^{B}, R^{C} or R^{D} comprises or is substituted with a phosphorus-containing moiety.

In certain embodiments of the compounds (5) described directly above, R^{G} is hydrogen or C₁₋₈ alkyl.

In certain other embodiments of the compounds (5) described directly above R^{D} is pyrazine.

In still other embodiments of the compounds (5) described directly above, ZR^{B} and R^{C} are each independently one of hydrogen, C₁₋₈ alkyl, C₃₋₁₀ alkyl, C₆₋₁₀ aryl or —CH₂R^{H}, wherein R^{H}, for each occurrence, is independently one of C₆₋₁₀ aryl, C₆₋₁₀aryl(C₁₋₆)alkyl, C₁₋₆alkyl(C₆₋₁₀)aryl, C₃₋₁₀cycloalkyl, C₁₋₈alkoxy, or C₁₋₈alkylthio, where the ring portion of any of the foregoing aryl, aralkyl, or alkaryl groups of ZR^{B}, R^{C} or R^{H} can optionally be substituted by one or two substituents independently selected from the group consisting of C₁₋₆alkyl, C₃₋₈cycloalkyl, C₁₋₆alkyl(C₃₋₈)cycloalkyl, C₂₋₈alkenyl, C₂₋₈alkynyl, cyano, amino, C₁₋₆alkylamino, di(C₁₋₆)alkylamino, benzylamino, dibenzylamino, nitro, carboxy, carbo(C₁₋₆)alkoxy, trifluoromethyl, halogen, C₁₋₆alkoxy, C₆₋₁₀aryl, C₆₋₁₀aryl(C₁₋₆)alkyl, C₆₋₁₀aryl(C₁₋₆)alkoxy, hydroxy, C₁₋₆alkylthio, C₁₋₆alkylsulfinyl, C₁₋₆alkylsulfonyl, C₆₋₁₀arylthio, C₆₋₁₀arylsulfinyl, C₆₋₁₀arylsulfonyl, C₆₋₁₀aryl, C₁₋₆alkyl(C₆₋₁₀)aryl, and halo(C₆₋₁₀)aryl.

In certain other embodiments, for compounds (5) described directly above, ZR^{B} is C₁₋₁₂ alkyl.

In still other embodiments, for compounds (5) described directly above, ZR^{B} is C₁₋₆ alkyl.

In yet other embodiments, for compounds (5) described directly above, ZR^{B} is C₄alkyl.

In still other embodiments, for compounds (5) described directly above, ZR^{B} is isobutyl.

In certain other embodiments, for compounds (5) described directly above, R^{C} is one of isobutyl, 1-naphthylmethyl, 2-naphthylmethyl, benzyl, 4-fluorobenzyl, 4-hydroxybenzyl, 4-(benzyloxy)benzyl, benzylnaphthylmethyl, or phenethyl.

In other embodiments, for compounds (5) described directly above, R^{D} is one of quinoline, pyridine, pyrazine, furan, or N-morpholine.

Another class of compounds of special interest includes those compounds wherein wherein R^{D} is an aryl moiety optionally substituted with one or more occurrences of R^{F} and R³, and R^{C} is a benzyl moiety optionally substituted with one or more occurrences of R^{E} and R³, and the compound has the structure (6):

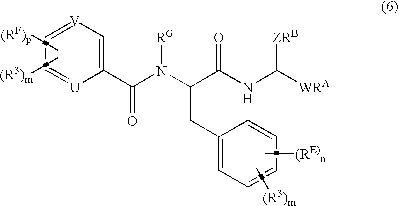

wherein each occurrence of R³, Y, R¹, G, M and x is as defined previously or in the various classes and subclasses herein;

at least one of R^{A}, R^{B}, R^{E} or R^{F} comprises or is substituted with a phosphorus-containing moiety;

V and U are each independently N or CR³;

n is 0-3; p is 0-3; m is 0-3, and the sum of m+p or n+m is an integer from 0-5.

In certain embodiments of the compounds (6) described directly above, R^{G} is hydrogen or C₁₋₈ alkyl.

In still other embodiments of the compounds (6) described directly above, ZR^{B} is hydrogen, C₁₋₈ alkyl, C₃₋₁₀ alkyl, C₆₋₁₀ aryl or —CH₂R^{H}, wherein R^{H}, for each occurrence, is independently one of C₆₋₁₀ aryl, C₆₋₁₀aryl(C₁₋₆)alkyl, C₁₋₆alkyl(C₆₋₁₀)aryl, C₃₋₁₀cycloalkyl, C₁₋₈alkoxy, or C₁₋₈alkylthio, where the ring portion of any of the foregoing aryl, aralkyl, or alkaryl groups of ZR^{B} or R^{H} can optionally be substituted by one or two substituents independently selected from the group consisting of C₁₋₆alkyl, C₃₋₈cycloalkyl, C₁₋₆alkyl(C₃₋₈)cycloalkyl, C₂₋₈alkenyl, C₂₋₈alkynyl, cyano, amino, C₁₋₆alkylamino, di(C₁₋₆)alkylamino, benzylamino, dibenzylamino, nitro, carboxy, carbo(C₁₋₆)alkoxy, trifluoromethyl, halogen, C₁₋₆alkoxy, C₆₋₁₀aryl, C₆₋₁₀aryl(C₁₋₆)alkyl, C₆₋₁₀aryl(C₁₋₆)alkoxy, hydroxy, C₁₋₆alkylthio, C₁₋₆alkylsulfinyl, C₁₋₆alkylsulfonyl, C₆₋₁₀arylthio, C₆₋₁₀arylsulfinyl, C₆₋₁₀arylsulfonyl, C₆₋₁₀aryl, C₁₋₆alkyl(C₆₋₁₀)aryl, and halo(C₆₋₁₀)aryl.

In certain other embodiments, for compounds (6) described directly above, ZR^{B} is C₁₋₁₂ alkyl.

In still other embodiments, for compounds (6) described directly above, ZR^{B} is C₁₋₆ alkyl.

In yet other embodiments, for compounds (6) described directly above, ZR^{B} is C₄alkyl.

In still other embodiments, for compounds (6) described directly above, ZR^{B} is isobutyl.

Still another class of compounds of special interest includes those compounds wherein wherein R^{D} is an aryl moiety optionally substituted with one or more occurrences of R^{F} and R³, and R^{C} is a benzyl moiety optionally substituted with one or more occurrences of R^{E} and R³, ZR^{B} is isobutyl, and the compound has the structure (7):

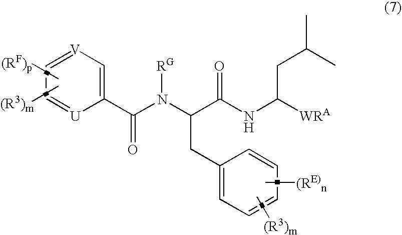

wherein each occurrence of R³, Y, R¹, G, M and x is as defined previously or in the various classes and subclasses herein; at least one of R^{A}, R^{E} or R^{F} comprises or is substituted with a phosphorus-containing moiety;

V and U are each independently N or CR³;

n is 0-3;

p is 0-3; and

m is 0-3, and the sum of m+p or n+m is an integer from 0-5.

A number of important subclasses of each of the foregoing classes deserve separate mention. These subclasses include those in which:

(i) only one of R^{A}, R^{B}, R^{C}, R^{D}, R^{E} or R^{F} comprises or is substituted with a phosphorus-containing moiety;

(ii) two or more of R^{A}, R^{B}, R^{C}, R^{D}, R^{E} or R^{F} comprise or are substituted with a phosphorus-containing moiety.

(iii) one or more occurrences of R^{A}, R^{B}, R^{C}, R^{D}, R^{E} or R^{F} comprises or is substituted with a phosphorus-containing moiety of Series I, above.

(iv) one or more occurrences of R^{A}, R^{B}, R^{C}, R^{D}, R^{E} or R^{F} comprises or is substituted with a phosphorus-containing moiety of Series Ia:, above.

(v) one or more occurrences of R^{A}, R^{B}, R^{C}, R^{D}, R^{E} or R^{F} comprises or is substituted with a phosphorus-containing moiety of Series Ib:, above.

(vi) compounds having phosphorus-containing moieties of Series I, Ia or Ib, wherein Y is O;

(vii) W is a covalent bond and R^{A} is B(OH)₂;

(viii) W is a lower alkyl moiety and R^{A} is a phosphorus-containing moiety;

(ix) ZR^{B} is a linear or branched, cyclic or acyclic, substituted or unsubstituted alkyl or other aliphatic moiety;

(x) ZR^{B} is C₁₋₁₂ alkyl, C₁₋₆ alkyl, C₄alkyl, or isobutyl;

(xi) ZR^{B} is, —CH₂(cyclopentyl), —CH₂(cyclohexyl), —CH₃, —CH₂CH₃, —CH₂CH₂CH₃, —CH₂CH₂CH₂CH₃, or —CH₂CH(CH₃)(CH₃);

(xii) Z is a lower alkyl or other aliphatic moiety and R^{B} is a phosphorus-containing moiety;

(xiii) R^{C} is a substituted or unsubstituted aryl, alkylaryl, heteroaryl, or alkylheteroaryl moiety;

(xiv) R^{D} is a substituted or unsubstituted aryl, alkylaryl, heteroaryl, or alkylheteroaryl moiety;

(xv) either or both of R^{C} and R^{D} comprise any one of the following structures:

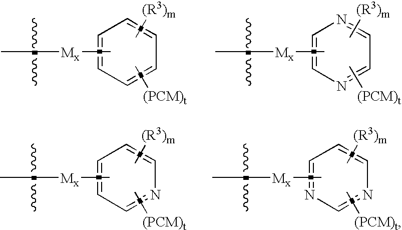

wherein each occurrence of R³, Y, R¹, G, M and x is as defined previously or in the various classes and subclasses herein;

PCM is a phosphorus-containing moiety of Series I, Series Ia or Series Ib; and

m and t are each independently an integer from 0-3, and the sum of m+t is an integer from 0-5;

(xvi) either or both of R^{C} and R^{D} comprise any one of the following structures:

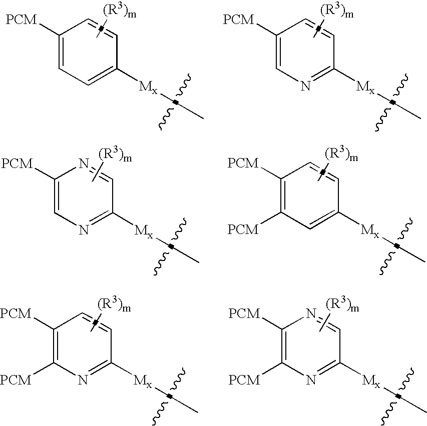

wherein each occurrence of R³, Y, R¹, G, M and x is as defined previously or in the various classes and subclasses herein;

m is an integer from 0-3; and

PCM is a phosphorus-containing moiety of Series I, Series Ia or Series Ib;

(xvii) either or both of R^{C} or R^{D} comprise any one of the following structures:

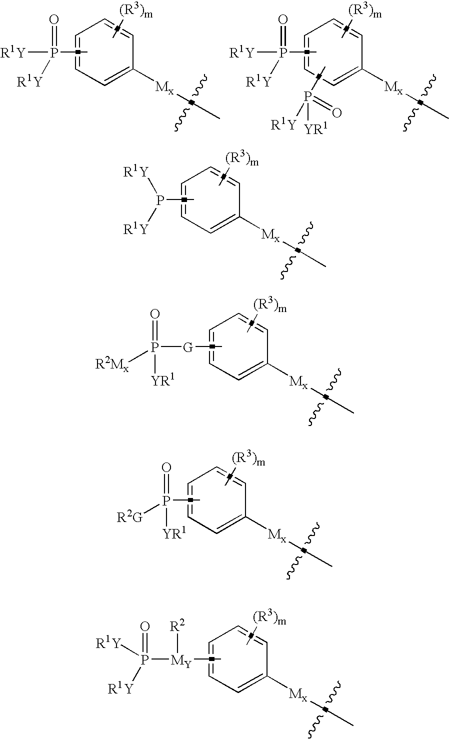

wherein each occurrence of R³, Y, R¹, G, M and x is as defined previously or in the various classes and subclasses herein; and

m is an integer from 0-3;

(xviii) R¹ is a substituted or unsubstituted alkyl moiety, or, except in YR¹ moieties in which Y is a covalent bond, R¹ may also be H;

(xix) at least one of R^{A}, R^{B}, R^{C}, R^{D}, R^{E} and R^{F} comprises or is substituted with:

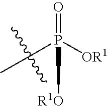

wherein each R¹ is independently H, alkyl, arylalkyl, aryl or a prodrug moiety;

(xx) at least one of R^{A}, R^{B}, R^{C}, R^{D}, R^{E} and R^{F} comprises or is substituted with:

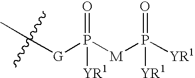

wherein each R¹ is independently H, alkyl, arylalkyl, aryl or a prodrug moiety;

(xxi) at least one of R^{A}, R^{B}, R^{C}, R^{D}, R^{E} and R^{F} comprises or is substituted with:

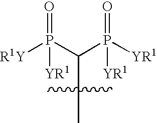

wherein each R¹ is independently H, alkyl, arylalkyl, aryl or a prodrug moiety;

(xxii) at least one of R^{A}, R^{B}, R^{C}, R^{D}, R^{E} and R^{F} comprises or is substituted with:

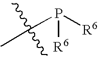

wherein each R⁶ is independently alkyl, arylalkyl, aryl or a prodrug moiety;

(xxiii) at least one of R^{A}, R^{B}, R^{C}, R^{D}, R^{E} and R^{F} comprises or is substituted with:

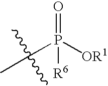

wherein R¹ is H, alkyl, arylalkyl or a prodrug moiety and R⁶ is alkyl, arylalkyl, aryl or a prodrug moiety;

(xxiv) at least one of R^{A}, R^{B}, R^{C}, R^{D}, R^{E} and R^{F} comprises or is substituted with:

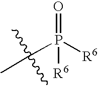

wherein each R⁶ is independently alkyl, arylalkyl, aryl or a prodrug moiety;

(xxv) at least one of R^{A}, R^{B}, R^{C}, R^{D}, R^{E} and R^{F} comprises or is substituted with:

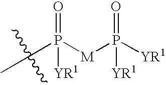

wherein each R¹ is H, alkyl, arylalkyl or a prodrug moiety, and Y and M are as defined previously;

(xxvi) at least one of R^{A}, R^{B}, R^{C}, R^{D}, R^{E} and R^{F} comprises or is substituted with:

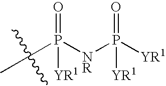

wherein each R¹ is independently H, alkyl, arylalkyl, aryl or a prodrug moiety and R is aliphatic, heteroaliphatic, aryl, or heteroaryl;

(xxvii) at least one of R^{A}, R^{B}, R^{C}, R^{D}, R^{E} and R^{F} comprises or is substituted with:

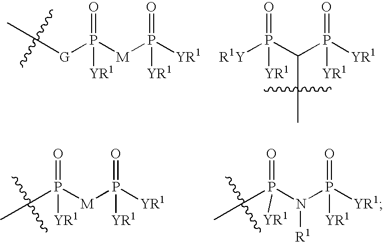

(xxviii) R^{G} is hydrogen;

(xxix) R^{G} is lower alkyl;

(xxx) R³ is hydrogen; and

(xxxi) ZR^{B} and R^{C} may each independently be hydrogen, C₁₋₈ alkyl, C₃₋₁₀ alkyl, C₆₋₁₀ aryl or —CH₂R^{H}, wherein R^{H}, for each occurrence, is independently one of C₆₋₁₀ aryl, C₆₋₁₀aryl(C₁₋₆)alkyl, C₁₋₆alkyl(C₆₋₁₀)aryl, C₃₋₁₀cycloalkyl, C₁₋₈alkoxy, or C₁₋₈alkylthio, where the ring portion of any of the foregoing aryl, aralkyl, or alkaryl groups of ZR^{B}, R^{C} or R^{H} can optionally be substituted by one or two substituents independently selected from the group consisting of C₁₋₆alkyl, C₃₋₈cycloalkyl, C₁₋₆alkyl(C₃₋₈)cycloalkyl, C₂₋₈alkenyl, C₂₋₈alkynyl, cyano, amino, C₁₋₆alkylamino, di(C₁₋₆)alkylamino, benzylamino, dibenzylamino, nitro, carboxy, carbo(C₁₋₆)alkoxy, trifluoromethyl, halogen, C₁₋₆alkoxy, C₆₋₁₀aryl, C₆₋₁₀aryl(C₁₋₆)alkyl, C₆₋₁₀aryl(C₁₋₆)alkoxy, hydroxy, C₁₋₆alkylthio, C₁₋₆alkylsulfinyl, C₁₋₆alkylsulfonyl, C₆₋₁₀arylthio, C₆₋₁₀arylsulfinyl, C₆₋₁₀arylsulfonyl, C₆₋₁₀aryl, C₁₋₆alkyl(C₆₋₁₀)aryl, and halo(C₆₋₁₀)aryl.

As the reader will appreciate, compounds of particular interest include, among others, those which share the attributes of one or more of the foregoing subclasses.

Some of those subclasses are illustrated by the following sorts of compounds:

I. Compounds of the Formula:

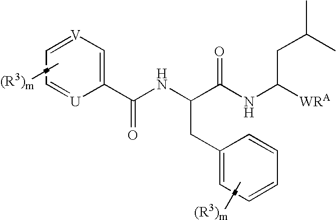

or a pharmaceutically acceptable derivative thereof,

wherein W is —(CH₂)ₛ wherein s is an integer from 0-6;

V and U are each independently CR₃ or N;

R_{A} is a phosphorus-containing moiety of Series I, Ia or Ib;

wherein each occurrence of R³, Y, R¹, G, M and x is as defined previously in connection with Sries I or in the various classes and subclasses herein; and

m is an integer from 0-3.

In certain embodiments for compounds described directly above, each occurrence of m is 0 and V and U are each N and the compound has the structure:

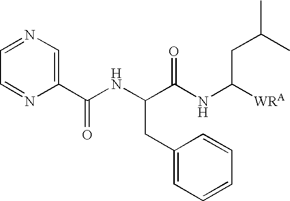

wherein R^{A} is a phosphorus-containing moiety of Series Ia.

II. Compounds of the Formula:

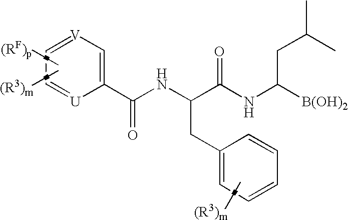

or a pharmaceutically acceptable derivative thereof,

wherein V and U are each independently CR₃ or N;

R^{F} is a phosphorus-containing moiety of Series I, Ia or Ib;

wherein each occurrence of R³, Y, R¹, G, M and x is as defined previously in connection with Series I or in the various classes and subclasses herein;

p is 1 or 2; and m is an integer from 0-3.

In certain embodiments, for compounds described directly above, each occurrence of m is 0 and V and U are each N and the compound has the structure:

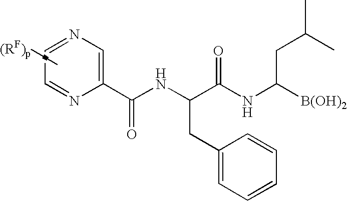

wherein R^{F} is a phosphorus-containing moiety of Series Ia and p is 1 or 2.

iii. Compounds of the Formula:

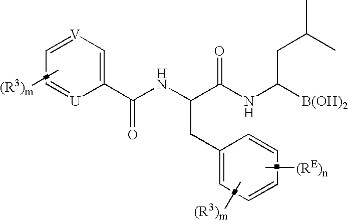

or a pharmaceutically acceptable derivative thereof,

wherein V and U are each independently CR₃ or N;

R^{E} is a phosphorus-containing moiety of Series I, Ia or Ib;

each wherein each occurrence of R³, Y, R¹, G, M and x is as defined previously in connection with Series I or in the various classes and subclasses herein;

n is 1 or 2; and m is an integer from 0-3.

In certain embodiments for compounds described directly above, each occurrence of m is 0, V and U are each N and the compound has the structure:

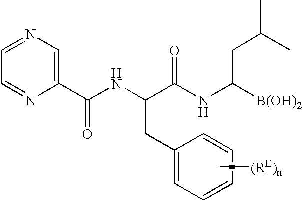

wherein R^{E} is a phosphorus-containing moiety of Series Ia and n is 1 or 2.

IV. Compounds having the Formula:

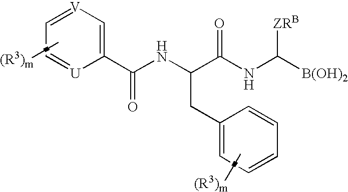

or a pharmaceutically acceptable derivative thereof,

wherein Z is —(CH₂), wherein s is an integer from 0-6;

V and U are each independently CR₃ or N;

R^{B} is a phosphorus-containing moiety of Series I, Ia or Ib;

wherein wherein each occurrence of R³, Y, R¹, G, M and x is as defined previously in connection with Series I or in the various classes and subclasses herein; and

each occurrence of m is an integer from 0-3.

In certain embodiments for the compounds as described directly above, each occurrence of m is 0, V and U are each N, and the compound has the structure:

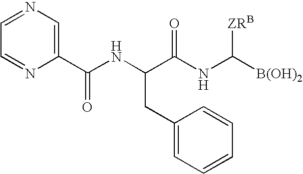

wherein R^{B} is a phosphorus-containing moiety of Series Ia.

It will be appreciated that some of the foregoing compounds can exist in various isomeric forms. The invention encompasses the compounds as individual isomers substantially free of other isomers and, alternatively, as mixtures of various isomers, e.g. racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of this invention which are of particular interest include those which measurably or detectably

bind to bone preferentially over other tissues,

improve the balance of bone growth relative to bone resorption (in favor of bone growth) in any scientifically acceptable animal model,

exibit a cytotoxic or growth inhibitory effect on cancer cell lines maintained in vitro or in animal studies using a scientifically acceptable cancer cell xenograft model;

inhibit the accelerated breakdown of muscle proteins;

reduce activity of NF-kB;

reduce the rate of degradation of p53 protein;

inhibit cyclin degradation;

inhibit antigen presentation;

inhibit replication of HIV in a mammal;

inhibit cytolytic immune responses; and

inhibit proteasome function in a mammal.

This invention also provides a pharmaceutical preparation comprising at least one of the foregoing compounds or a pharmaceutically acceptable derivative thereof, as inhibitors of bone resorption by osteoclasts, as inhibitors of growth of tumors or other cancers (including among others, multiple myeloma; cancer of the bone as well as cancers which have spread to bone; as well as pancreatic, colon, lung, breast, prostate and ovarian cancers), as inhibitors of tumor metastasis, as inhibitors of NF-kB activity, as inhibitors of cyclin degradation, as inhibitors of HIV replication, and as inhibitors of cytolytic immune responses, and at least one pharmaceutically acceptable excipient or additive. Preferably the excipient or additive is pharmaceutically innocuous.

The invention further provides a method for inhibiting bone resorption, for inhibiting tumor or other cancerous growth and/or metastasis, and for the treatment or prevention of diseases or undesirable conditions as described herein. The method involves administering a therapeutically effective amount of the compound or a pharmaceutically acceptable derivative thereof to a human or animal in need of it.

3. Compounds and Definitions

This invention provides a new family of compounds with a range of biological properties. Compounds of this invention have biological activities relevant for the treatment of diseases including bone related disorders, and proliferative diseases, including multiple myeloma and other cancers, to name a few. More generally, the compounds are useful as inhibitors of the proteaseome function and thus have a number of practical therapeutic and prophylactic indications as described in more detail herein.

Compounds of this invention include those specifically set forth above and described herein, and are illustrated in part by the various classes, subgenera and species disclosed elsewhere herein.

It will be appreciated by one of ordinary skill in the art that asymmetric centers may exist in the compounds of the present invention. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, a mixtures of stereoisomers or diastereomers are provided.

Additionally, the present invention provides pharmaceutically acceptable derivatives of the inventive compounds, and methods of treating a subject using these compounds, pharmaceutical compositions thereof, or either of these in combination with one or more additional therapeutic agents. The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety which is susceptible to removal in vivo yielding the parent molecule as the pharmacologically active species. An example of a pro-drug is an ester which is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other funcational groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

Additionally, as described herein, esters of COOH and B(OH)₂ are also encompassed. For example, esters of B(OH)₂ include, but are not limited to compounds of the type —B(R_{y})(R_{y′}), where R_{y} and R_{y′} are each independently alkyl, alkoxy, aryloxy, or together form a moiety derived from a dihydroxy compound having at least two hydroxy groups separated by at least two connecting atoms in a chain or a ring, said chain or ring comprising carbon atoms, and optionally, a heteroatom or heteroatoms which can be N, S or O. In certain exemplary embodiments, R_{y} and R_{y′} are both C₁₋₆alkoxy or C₆₋₁₀ aryloxy, or together R_{y} and R_{y′} form a moiety derived from a dihydroxy compound selected from the group consisting of pinacol, perfluoropinacol, pinanediol, ethylene glycol, diethylene glycol, 1,2-cyclohexanediol, 1,3-propanediol, diethylene glycol, 1,2-cyclohexanediol, 1,3-propanediol, 2,3-butanediol, glycerol or diethanolamine.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of bone related disorders, cancer, disorders related to increases in vascular permeability, and/or disorders related to call signalling. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups having 1-6 carbon atoms.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alkoxy”, or “thioalkyl” as used herein refers to an alkyl group, as previously defined, attached to the parent molecular moiety through an oxygen atom or through a sulfur atom. In certain embodiments, the alkyl group contains 1-20 alipahtic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Examples of suitable substituents include halogen, —YR¹ (i.e., including —R¹, —OR¹, —SR¹ and —NR¹R^{1′}), —Y—C(═O)R¹, —Y—C(═O)OR¹, —Y—C(═O)NR¹R^{1′}, —Y—C(═NR^{1′})NR¹R^{1″}, —COCOR¹, —COMCOR¹), a phosphorus-containing moiety, —CN, —S(═O)R¹, —SO₂R¹, —SO₂NR¹R^{1′}, —NO₂, —NR¹SO₂R^{1′} and —NR^{1″}SO₂NR¹R^{1′}. To illustrate further, substituents in which Y is NR¹ thus include among others, —NR¹C(═O)R^{1′}, —NR¹C(═O)NR^{1′}, —NR¹C(═O)OR^{1′}, and —NR¹C(═NH)NR^{1′}. Note that each R¹ substituent may itself be substituted or unsubstituted (e.g. non-limiting illustrations of an R¹ moiety include -alkylhalo such as chloromethyl or trichloromethyl; -alkoxyalkyl such as methoxyethyl-; mono-, di- and tri-alkoxyphenyl; methylenedioxyphenyl or ethylenedioxyphenyl; halophenyl; and alkylamino). Additional illustrative examples include 1,2-methylene-dioxy, 1,2-ethylenedioxy, protected OH (such as acyloxy)), phenyl, substituted phenyl, -O-phenyl, -O-(substituted) phenyl, -benzyl, substituted benzyl, —O-phenethyl (i.e., —OCH₂CH₂C₆H₅), —O-(substituted)phenethyl, —C(O)CH₂C(O)R¹, —CO₂R¹, —C(═O)R¹ (i.e., acyl in cases in which R¹ is aliphatic, aroyl in cases in which R¹ is aryl and heteroaroyl in cases in which R¹ is heteroaryl), —C(═O)NR¹R^{1′}, —OC(═O)NR¹R^{1′}, —C(═NH)NR¹R^{1′}, and —OC(═NH)NR¹R^{1′}. Further examples of substituents include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, and haloalkyl groups.

Some examples of substituents thus include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; and —NRₓ(CO)Rₓ moieties wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. In addition, it will be appreciated that compounds can be substituted with one or more phosphorus-containing moieties, as defined above, and herein. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl and the like. In certain embodiments of the present invention, the term “heteroaryl”, as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one, two or three of the hydrogen atoms thereon independently with any one or more of the following moieties including, but not limited to: aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. In addition, it will be appreciated that compounds can be substituted with one or more phosphorus-containing moieties, as defined above, and herein. Additional examples of generally applicable substitutents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or hetercyclic moieties, may optionally be substituted with substituents including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. In addition, it will be appreciated that compounds can be substituted with one or more phosphorus-containing moieties, as defined above, and herein. Additional examples of generally applicable substitutents are illustrated by the specific embodiments shown in the Examples that are described herein.

The term “heteroaliphatic”, as used herein, refers to aliphatic moieties which contain one or more oxygen, sulfur, nitrogen, phosphorous or silicon atoms, e.g., in place of carbon atoms. Heteroaliphatic moieties may be branched, unbranched or cyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. In addition, it will be appreciated that compounds can be substituted with one or more phosphorus-containing moieties, as defined above, and herein. Additional examples of generally applicable substitutents are illustrated by the specific embodiments shown in the Examples that are described herein.

The terms “halo” and “halogen” as used herein refer to an atom selected from F, Cl, Br and I.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6- or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. In addition, it will be appreciated that compounds can be substituted with one or more phosphorus-containing moieties, as defined above, and herein. Additional examples of generally applicable substitutents are illustrated by the specific embodiments shown in the Examples which are described herein.

“Phosphorus-containing moiety” or “PCM”: As used herein, the phrase, “phosphorus-containing moiety” or “PCM” includes, but is not limited to, phosphites, phosphonites, phosphenites, phosphines, phosphates, phosphonates, phosphenates, phosphine oxides, bisphosphonates, thiophosphates, thiophosphonates, thiophosphenates, thiophosphine oxides, mono- or (where permitted) di- or tri-amides and esters of any of the foregoing as well as the phosphorus-containing moieties disclosed in Series I, Ia, and Ib, and in the accompanying text and in the various classes, subclasses, and species of compounds disclosed herein.

4. Prodrugs

Numerous suitable prodrug moieties, and information concerning their selection, synthesis and use are well known, beginning with lower alkyl esters of phosphonates and related moieties. Other prodrug moieties of interest include the following:

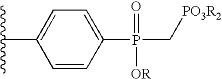

| R | |
|---|---|
| 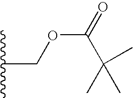 | Atack, J. R. et al. J. of Pharmacology and Experimental Therapeutics 1994, 270, 70. |
| 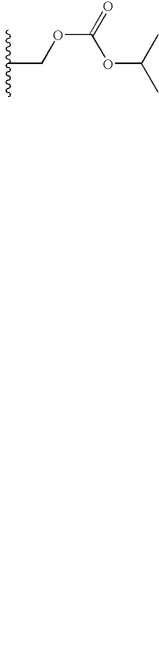 | Arimilli, M. N., et al. Antiviral Chemistry & Chemotherapy 1997, 8, 557. |
| 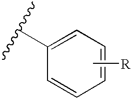 | Serafinowska, H. T., et el. J. Med. Chem. 1995, 35, 1372. |
| 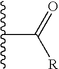 | Ahlmark, M., J. Med. Chem. 1999, 42, 1473. |
| 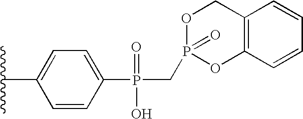 | Meier, C., et al. J. Med. Chem. 1998, 41, 1417. |

Review: Krise, J. P., Stella, V. J. Advanced Drug Delivery Reviews 1996, 19, 287. and references cited therein.

Other prodrug moieties of interest that can be attached to primary or secondary amine-containing functionality include the following:

| | |
|---|---|
| 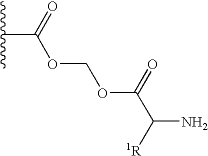 | For the synthesis of the prodrug groups, see Borchardt, R. T. et. al., J. Org. Chem. 1997, 43, 3641-3652. |
| R¹ = all natural, | |
| unnatural amino acids | |
| 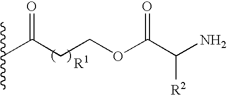 | For the synthesis of the prodrug groups, see Zhou, X-X. et. al., PCT WO 99/51613. |
| R¹ = C1-C4 alkyl, cycloalkyl, oxyalkyl, | |
| aminoalkyl, etc. | |
| R² = all natural, unnatural amino acids | |
| 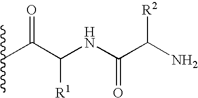 | For the synthesis of the prodrug groups, see Ezra, A. et. al., J. Med. Chem. 2000, 43, 3641-3652. |
| R¹, R² = all natural, unnatural amino acids | |

5. Uses, Formulations, Administration

Pharmaceutical Compositions

As discussed above the present invention provides novel compounds that are useful for the treatment or prevention of a variety of disorders. In general, in one embodiment, compounds of the invention are useful for reducing the rate of proteasome dependent intracellular protein breakdown, such as reducing the rate of muscle protein degradation, reducing the rate of degradation of p53 protein, and inhibiting cyclin degradation, and for inhibiting the activity of NF-kB in a cell. As such, the inventive compounds are useful for treating specific conditions in animals that are mediated or exacerbated, directly or indirectly, by proteasome functions. These conditions include inflammatory conditions, such as tissue rejection, organ rejection, arthritis, infection, dermatoses, inflammatory bowel disease, asthma, osteoporosis, osteoarthritis and autoimmune disease such as lupus and multiple sclerosis; cell proliferative diseases, such as cancer, psoriasis and restenosis; and accelerated muscle protein breakdown that accompanies various physiological and pathological states and is responsible to a large extent for the loss of muscle mass (atrophy) that follows nerve injury, fasting, fever, acidosis, and certain endocrinopathies. In other embodiments, compounds of the invention are also useful for the treatment of bone-related disorders and, in particular, for the inhibition of osteoclast activity and to tilt the balance of bone resorption and bone growth positively, i.e., away from net bone loss. It is currently preferred that the compounds used for such indications be compounds of this invention that have one or more free OH or SH groups on or adjacent to the phosphorus-containing moiety or moieties which characterize these compounds. Thus, such compounds will often contain one or more —YR¹ moieties in which R¹ is H. Alternatively, prodrugs of such compounds may also be chosen. In yet another embodiment, compounds of the invention are also useful in the inhbition of infection by the hepatits C virus and are useful for inhibition of HIV replication.

Accordingly, in another aspect of the present invention, pharmaceutical compositions are provided, wherein these compositions comprise any one of the compounds as described herein, and optionally comprise a pharmaceutically acceptable carrier. In certain embodiments, these compositions optionally further comprise one or more additional therapeutic agents, or an approved agent for the treatment of disorders as discussed in more detail herein.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof, e.g., a prodrug.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters includes formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-viral compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Uses of Compounds of the Invention

As discussed herein, the compounds of the present invention are useful for the treatment or prevention of a variety of disorders, including, but not limited to the treatment of proliferative disorders such as cancer and the treatment of bone-related disorders. Additionally, various compounds of the invention can be used to inhibit osteoclast activity and to tilt the balance of bone resorption and bone growth positively, i.e., away from net bone loss.

Thus, compounds as described herein are useful for the treatment of bone disorders, proliferative disease, such as cancer, and other disorders as described above. In another aspect, methods for the treatment of these disorders is provided comprising administering to a subject in need thereof an effective amount of a compound of Formula I as described herein, or pharmaceutically acceptable derivatives thereof, and any of the classes and subclasses herein. The compounds may be formulated and administered using materials and methods known in the art for formulation and administration of Velcade (Bortezomib) and related compounds. See e.g. WO 02/059131. (e.g. with administration of 0.1 to 20 mg drug/m2 of patient, preferably 0.1-10 mg/m2, and in some cases 1.0-1.5 mg drug/m2 for Injection therapy. Therapy may be limited to a particular time course, e.g. 2-12 months, in some cases for up to 6 months, dosed intermittently, e.g. on days 1, 4, 8 and 11 of a 21-day cycle, e.g. for up to 6-12 cycles).

In certain embodiments of the present invention a “therapeutically effective amount” of the inventive compound or pharmaceutical composition is that amount effective for killing or inhibiting the growth of tumor cells, or is an amount that is effective for promoting or inhibiting osteoclast activity, which activity is believed to be involved in the effect of bone disorders, although the present invention is not intended to be bound by any particular theory. In general, the term “therapeutically effective amount” of the inventive compound or pharmaceutical composition is that amount that is effective for treating a particular disorder (e.g., eradication) or for ameliorating the symptoms or effects of said disorder (e.g., inhibition of HIV replication, or inhibition of tumor growth).

The compounds and compositions, according to the method of the present invention, may be administered using any effective amount and any route of administration. The exact amount required will vary from subject to subject, depending on the species, age, and general condition of the subject, the severity of the infection, the particular therapeutic agent, its mode of administration, and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As discussed above, in one aspect, the compounds of the present invention are useful as anticancer agents, and thus may be useful in the treatment of cancer, by effecting tumor cell death or inhibiting the growth of tumor cells. In general, the inventive anticancer agents are useful in the treatment of cancers and other proliferative disorders. By the term “treatment of cancer” or “treating cancer” is intended description of an activity of compounds of the present invention wherein said activity prevents or alleviates or ameliorates any of the specific phenomena known in the art to be associated with the pathology commonly known as “cancer.” The term “cancer” refers to the spectrum of pathological symptoms associated with the initiation or progression, as well as metastasis, of malignant tumors. By the term “tumor” is intended, for the purpose of the present invention, a new growth of tissue in which the multiplication of cells is uncontrolled and progressive. The tumor that is particularly relevant to the invention is the malignant tumor, one in which the primary tumor has the properties of invasion or metastasis or which shows a greater degree of anaplasia than do benign tumors. Thus, “treatment of cancer” or “treating cancer” refers to an activity that prevents, alleviates or ameliorates any of the primary phenomena (initiation, progression, metastasis) or secondary symptoms associated with the disease. Cancers that are treatable are broadly divided into the categories of carcinoma, lymphoma and sarcoma. Examples of carcinomas that can be treated by the composition of the present invention include, but are not limited to: adenocarcinoma, acinic cell adenocarcinoma, adrenal cortical carcinomas, alveoli cell carcinoma, anaplastic carcinoma, basaloid carcinoma, basal cell carcinoma, bronchiolar carcinoma, bronchogenic carcinoma, renaladinol carcinoma, embryonal carcinoma, anometroid carcinoma, fibrolamolar liver cell carcinoma, follicular carcinomas, giant cell carcinomas, hepatocellular carcinoma, intraepidermal carcinoma, intraepithelial carcinoma, leptomanigio carcinoma, medullary carcinoma, melanotic carcinoma, menigual carcinoma, mesometonephric carcinoma, oat cell carcinoma, squamal cell carcinoma, sweat gland carcinoma, transitional cell carcinoma, and tubular cell carcinoma. Sarcomas that can be treated by the composition of the present invention include, but are not limited to: amelioblastic sarcoma, angiolithic sarcoma, botryoid sarcoma, endometrial stroma sarcoma, ewing sarcoma, fascicular sarcoma, giant cell sarcoma, granulositic sarcoma, immunoblastic sarcoma, juxaccordial osteogenic sarcoma, coppices sarcoma, leukocytic sarcoma (leukemia), lymphatic sarcoma (lympho sarcoma), medullary sarcoma, myeloid sarcoma (granulocitic sarcoma), austiogenci sarcoma, periosteal sarcoma, reticulum cell sarcoma (histiocytic lymphoma), round cell sarcoma, spindle cell sarcoma, synovial sarcoma, and telangiectatic audiogenic sarcoma. Lymphomas that can be treated by the composition of the present invention include, but are not limited to: Hodgkin's disease and lymphocytic lymphomas, such as Burkitt's lymphoma, NPDL, NML, NH and diffuse lymphomas.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

For example, other therapies or anticancer agents that may be used in combination with the inventive anticancer agents of the present invention include surgery, radiotherapy (in but a few examples, g-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

As discussed above, in another aspect, the compounds of the present invention are useful in the selective treatment or prevention of bone disorders, and may effect treatment via inhibition of osteoclast activity, promotion of osteoblast activity, or promotion or inhibition of other cellular events necessary for healthy bone metabolism. In certain preferred embodiments, these compounds are useful for the treatment or prevention of diseases and conditions associated with bone metabolic disorders such as osteoclast overactivity.

The present invention therefore provides a method for the treatment, prophylaxis, and/or prevention of bone and other related disorders which method comprises the administration of an effective non-toxic amount of an inventive compound, or a pharmaceutically composition thereof. As mentioned above, although the inventive compounds effect treatment via several mechanisms, (i.e. inhibition of osteoclast activity, promotion of osteoblast activity, or regulation of other cellular events necessary for healthy bone metabolism), in certain preferred embodiments, these compounds are selective inhibitors of osteoclast activity.

It will be appreciated that, in addition to the treatment or prevention of osteoporosis, particularly osteoporosis associated with the peri and post menopausal conditions, the present invention also contemplates the treatment and prophylaxis or prevention of Paget's disease, hypercalcemia associated with bone neoplasms and other types of osteoporotic diseases and related disorders, including but not limited to involutional osteoporosis, Type I or postmenopausal osteoporosis, Type II or senile osteoporosis, juvenile osteoporosis, idiopathic osteoporosis, endocrine abnormality, hyperthyroidism, hypogonadism, ovarian agensis or Turner's syndrome, hyperadrenocorticism or Cushing's syndrome, hyperparathyroidism, bone marrow abnormalities, multiple myeloma and related disorders, systemic mastocytosis, disseminated carcinoma, Gaucher's disease, connective tissue abnormalities, osteogenesis imperfecta, homocystinuria, Ehlers-Danlos syndrome, Marfan's syndrome, Menke's syndrome, immobilization or weightlessness, Sudeck's atrophy, chronic obstructive pulmonary disease, chronic heparin administration, and chronic ingestion of anticonvulsant drugs.

The present invention additionally relates to a method of inhibiting (reducing or preventing) the accelerated or enhanced proteolysis that occurs in atrophying muscles and is known to be due to activation of a nonlysosomal ATP-requiring process in which ubiquitin plays a critical role.

Inhibition of the ATP-ubiquitin-dependent pathway is a new approach for treating the negative nitrogen balance in catabolic states. This can be effected through use of an inhibitor of the present invention, resulting in reduction of loss of muscle mass in conditions in which it occurs. Excessive protein loss is common in many types of patients, including individuals with sepsis, burns, trauma, many cancers, chronic or systemic infections, neuromotor degenerative disease, such as muscular dystrophy, acidosis, or spinal or nerve injuries. It also occurs in individuals receiving corticosteroids, and those in whom food intake is reduced and/or absorption is compromised. Moreover, inhibitors of the protein breakdown pathway could possibly be valuable in animals, e.g., for combating “shipping fever”, which often leads to a major weight loss in cattle or pigs.

The accelerated proteolysis evident in atrophy of skeletal muscles upon denervation or fasting is catalyzed by the nonlysosomal ATP-dependent degradative pathway. It has been shown that in a variety of catabolic states (e.g., denervation, fasting, fever, certain endocrinopathies or metabolic acidosis) muscle wasting is due primarily to accelerated protein breakdown and, in addition, that the increased proteolysis results from activation of the cytosolic ATP-ubiquitin-dependent proteolytic system, which previously had been believed to serve only in the rapid elimination of abnormal proteins and certain short-lived enzymes. The discovery that this pathway is responsible for the accelerated proteolysis in these catabolic states is based on studies in which different proteolytic pathways were blocked or measured selectively in incubated muscles, and the finding of increased mRNA for components of this pathway (e.g., for ubiquitin and proteasome subunits) and increased levels of ubiquitin-protein conjugates in the atrophying muscles. The nonlysosomal ATP-ubiquitin-dependent proteolytic process increases in muscle in these conditions and is responsible for most of the accelerated proteolysis that occurs in atrophying muscles. There is a specific increase in ubiquitin mRNA, induction of mRNA for proteasome and increased ubiquitinated protein content in atrophying muscles that is not seen in non-muscle tissue under the same conditions.

The inhibitors of the present invention can be used to reduce (totally or partially) the nonlysosomal ATP-dependent protein degradation shown to be responsible for most of the increased protein degradation that occurs during fasting, denervation, or disuse (inactivity), steroid therapy, febrile infection, and other conditions. The compounds can be administered alone or in combination with another inhibitor or an inhibitor of another pathway (e.g., a lysosomal or Ca²⁺-dependent pathway) responsible for loss of muscle mass.

As detailed herein, in yet another embodiment, compounds of the invention are also useful for the treatment of chronic or acute inflammation that is the result of transplantation rejection, arthritis, rheumatoid arthritis, infection, dermatosis, inflammatory bowel disease, asthma, osteoporosis, osteoarthritis and autoimmune disease. Additionally, inflammation associated with psoriasis and restenosis can also be treated. Specifically, NF-kB, by mediating events such as the production of cytokines and the induction and utilization of cell-surface adhesion molecules, is a central and coordinating regulator involved in immune responses.

The term “treatment of inflammation” or “treating inflammation” is intended to include the administration of compounds of the present invention to a subject for purposes which can include prophylaxis, amelioration, prevention or cure of an inflammatory response. Such treatment need not necessarily completely ameliorate the inflammatory response. Further, such treatment can be used in conjunction with other traditional treatments for reducing the inflammatory condition known to those of skill in the art.

The compounds of the invention can be provided as a “preventive” treatment before detection of an inflammatory state, so as to prevent the same from developing in patients at high risk for the same, such as, for example, transplant patients.

In another embodiment, efficacious levels of the compounds of the invention are administered so as to provide therapeutic benefits against the secondary harmful inflammatory effects of inflammation. By an “efficacious level” of a composition of the invention is meant a level at which some relief is afforded to the patient who is the recipient of the treatment. By an “abnormal” host inflammatory condition is meant an level of inflammation in the subject at a site which exceeds the norm for the healthy medical state of the subject, or exceeds a desired level. By “secondary” tissue damage or toxic effects is meant the tissue damage or toxic effects which occur to otherwise healthy tissues, organs, and the cells therein, due to the presence of an inflammatory response, including as a result of a “primary” inflammatory response elsewhere in the body.

Thus, the compounds are useful for treating such conditions as tissue rejection, arthritis, local infections, dermatoses, inflammatory bowel diseases, autoimmune diseases, etc. The proteasome inhibitors of the present invention can be employed to prevent the rejection or inflammation of transplanted tissue or organs of any type, for example, heart, lung, kidney, liver, skin grafts, and tissue grafts.

### Treatment Kits

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the substituted purine dosages, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Exemplification

### EXAMPLE 1

Certain Exemplary Compounds:

General Synthetic Overview

The practitioner has a well-established literature of chemistry to draw upon, in combination with the information contained in the many examples which follow, for guidance on synthetic strategies, protecting groups, and other materials and methods useful for the synthesis of the compounds of this invention, including compounds containing R substituents. The following references, and the references cited therein, may be of particular interest: In particular, provides useful background information which may be adapted to synthesis (including protecting groups), assays, formulation and use of compounds of this invention.

Various solution phase and solid phase syntheses are disclosed in detail in the examples that follow which provide interesting and helpful examples of many representative chemical transformations and total syntheses. Additional synthetic guidance is provided in U.S. Pat. No. 5,780,454 and U.S. Pat. No. 6,265,380, the entire contents of which are hereby incorporated by reference.

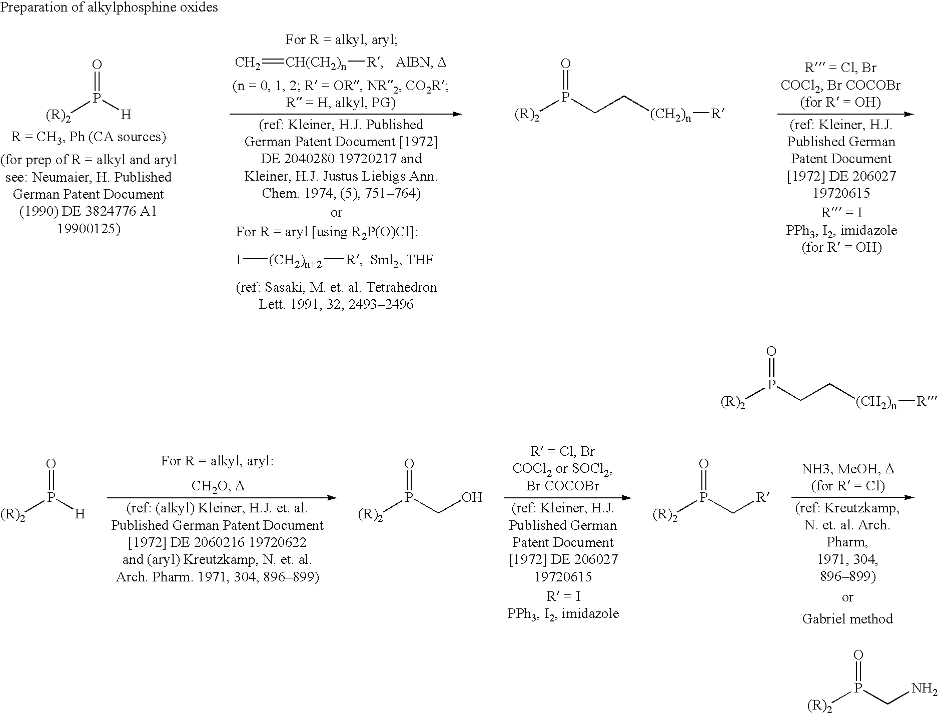

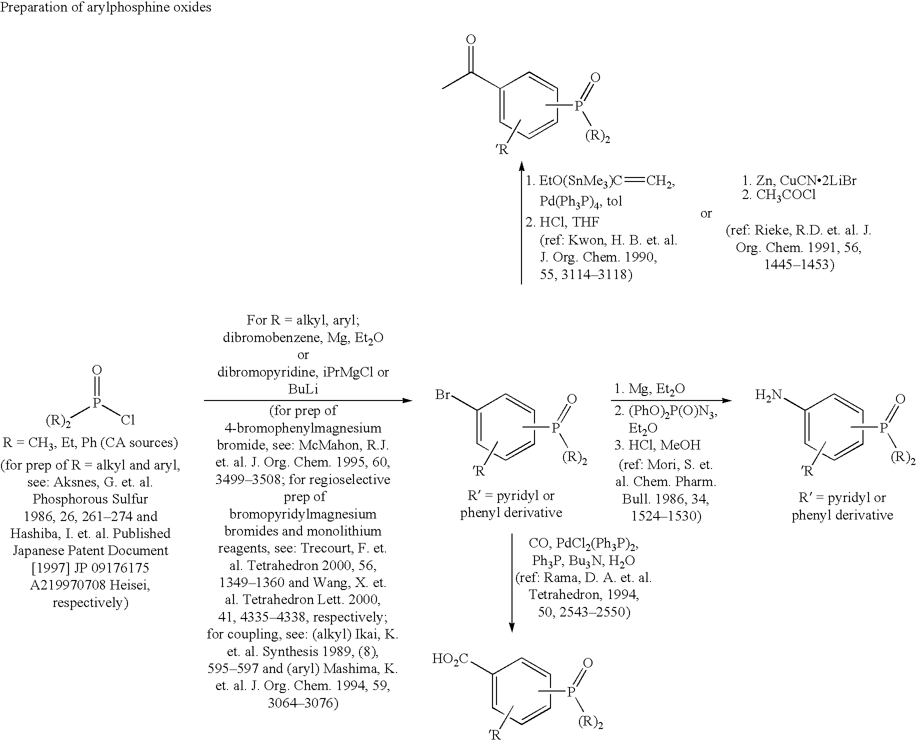

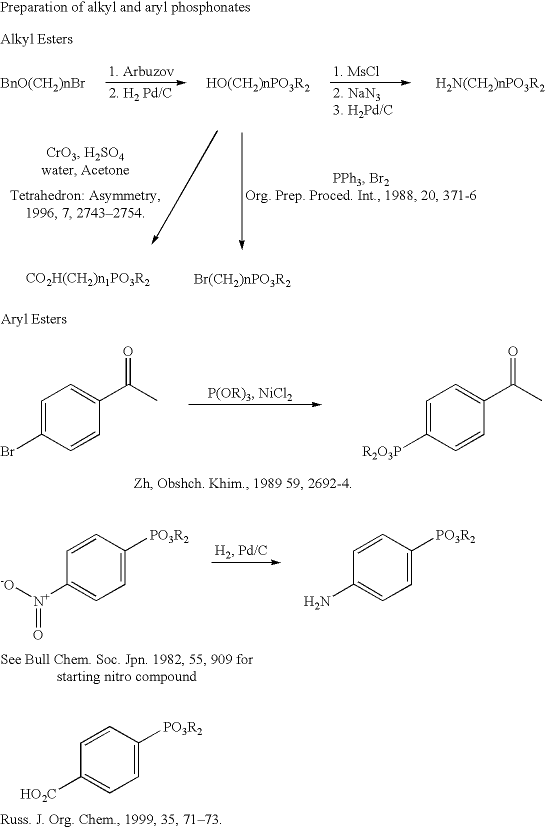

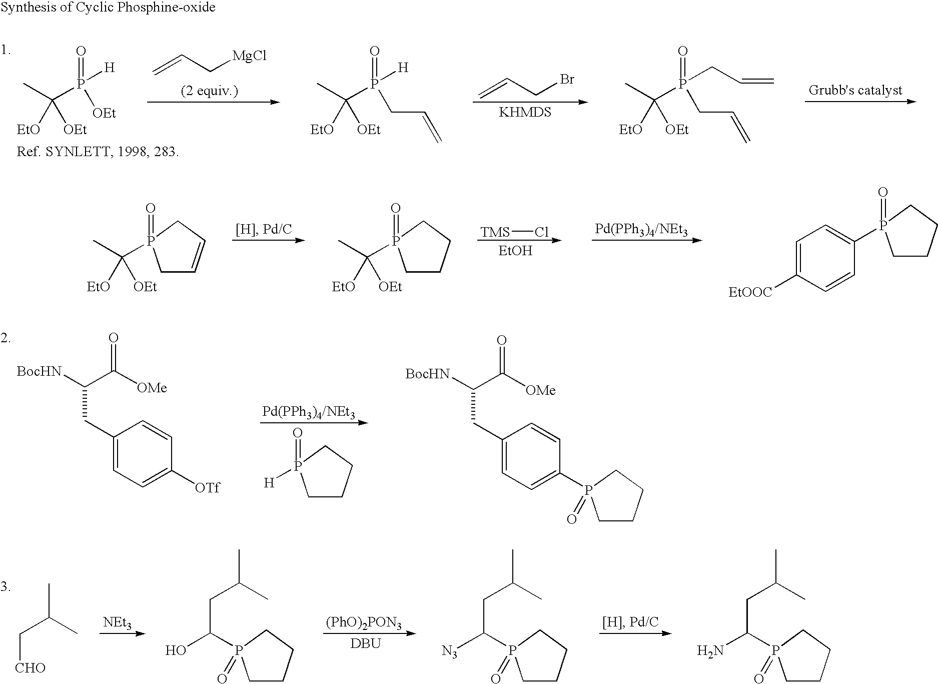

In addition to the phosphorus-containing moieties as described above and in PCT/US/34487, PCT/US/00/34417, 09/740,653, and 09/740,267, the entire contents of which are hereby incorporated by reference, certain other phosphorus-containing moieties, such as the described dialkyl phenyl phosphine oxide compounds can be synthesized according to the schemes outlined below:

4-(Dimethyl-phosphinoyl)-phenylamine hydrochloride

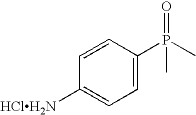

1-(Dimethyl-phosphinoyl)-4-fluoro-benzene

To a cooled (0° C.) flask containing 34.0 mL (2.0 M in Et₂O, 68.4 mmol) of 4-fluorophenylmagnesium bromide, under an atmosphere of N₂, was added a solution of dimethylphosphinic chloride (3.50 g, 31.1 mmol) in 84 mL of THF, dropwise via cannulation, over 20 min. The green reaction mixture was stirred at 0° C. for 1 h, then quenched at 0° C. with 30 mL of saturated NH₄Cl resulting in the formation of a white precipitate. The mixture was concentrated on a rotary evaporator and partitioned between EtOAc (200 mL) and H₂O (200 mL), upon which the layers were separated. The aqueous layer was extracted with EtOAc (3×100 mL) and the combined organics washed with brine, then dried over MgSO₄ and concentrated. The crude product was purified by silica gel flash chromatography (eluted with 5% MeOH/DCM) to provide 2.08 g of an off-white solid: ¹H NMR (300 MHz, DMSO-d₆) d 7.83 (m, 2H), 7.35 (td, J=8.9, 1.7 Hz, 2H), 1.66 (s, 3H), 1.61 (s, 3H). ³¹P NMR (121 MHz, DMSO-d₆) d 37.186. ¹⁹F NMR (282 MHz, DMSO-d₆) d-105.14.

1-(Dimethyl-phosphinoyl)-4-nitro-benzene

A sealed pressure flask, flushed with N₂, containing a mixture of 3.90 g (22.7 mmol) of 1-(Dimethyl-phosphinoyl)-4-fluoro-benzene and 6.0 g (113.3 mmol) of LiNO₂ (for prep see, W. C. Ball and H. H. Abram J. Chem. Soc. 1913, 103, 2130-2134) in 27 mL of DMPU (1,3-dimethyl-3,4,5,6-tetrahydro-2(1H)-pyrimidinone) was stirred at ambient temperature for 5-10 min (to dissolve fluoro compound) then heated at 190° C. for 3 days. The resulting dark brown solution was cooled to ambient temperature, diluted with 300 mL of brine, then extracted with EtOAc (10×100 mL) until the aqueous layer showed little or no evidence of product by HPLC. The combined organics were dried over MgSO₄ and concentrated. The excess DMPU was removed via short-path distillation (120° C./0.3 mm) to provide a semi-solid, which was dissolved in a minimum amount of iPrOH and purified by silica gel flash chromatography (eluted with 5% iPrOH/DCM, then 10% iPrOH/DCM, then 15% iPrOH/DCM) to provide 2.04 g of an yellow solid: ¹H NMR (300 MHz, DMSO-d₆) d 8.34 (dd, J=8.7, 1.9 Hz, 2H), 8.07 (dd, J=10.6, 8.8 Hz, 2H), 1.75 (s, 3H), 1.70 (s, 3H). ³¹P NMR (121 MHz, DMSO-d₆) d 37.89.

4-(Dimethyl-phosphinoyl)-phenylamine hydrochloride

A suspension of 1-(Dimethyl-phosphinoyl)-4-nitro-benzene (2.04 g, 10.2 mmol) and 10% palladium on carbon (0.411 g) in 103 mL of absolute EtOH containing 1.15 mL (11.4 mmol) of cock. HCl was flushed with H₂ and stirred at ambient temperature (H₂ balloon) for 2 h. The reaction mixture was filtered through Celite, the Celite washed with EtOH, and the combined filtrates concentrated to provide the crude product. Recrystallization from boiling iPrOH (10 mL) provided, after several crops, 1.17 g of an off-white solid:

¹H NMR (300 MHz, DMSO-d₆) d 7.61 (dd, J=11.1, 8.4 Hz, 2H), 7.04 (dd, J=8.4, 2.0 Hz, 2H), 1.64 (s, 3H), 1.60 (s, 3H). ³¹P NMR (121 MHz, DMSO-d₆) d 39.299.

Synthesis of Exemplary Compounds:

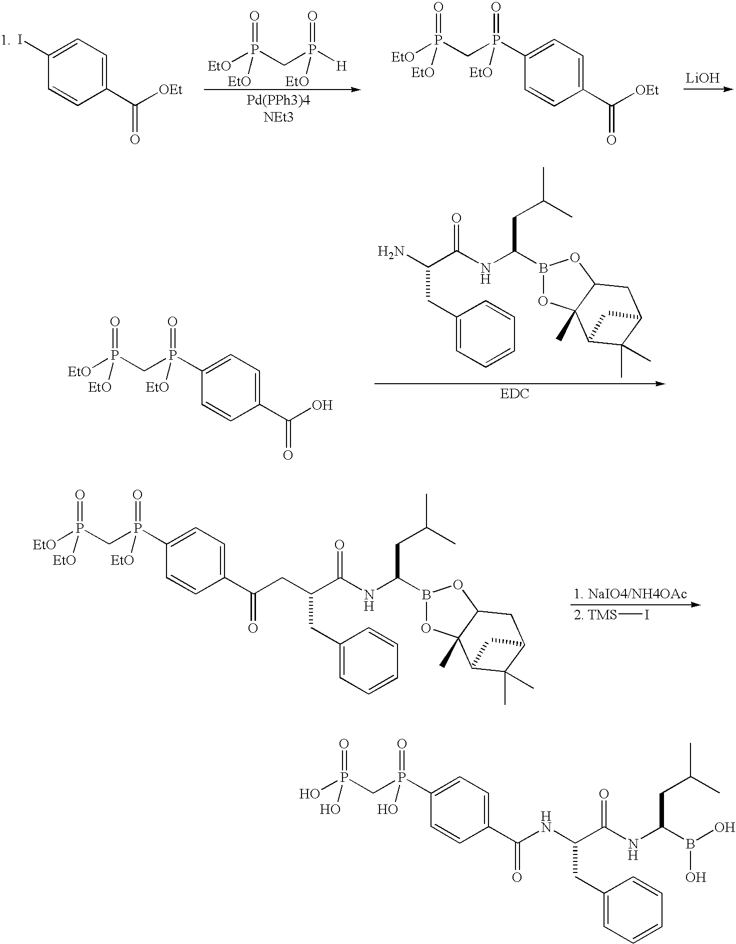

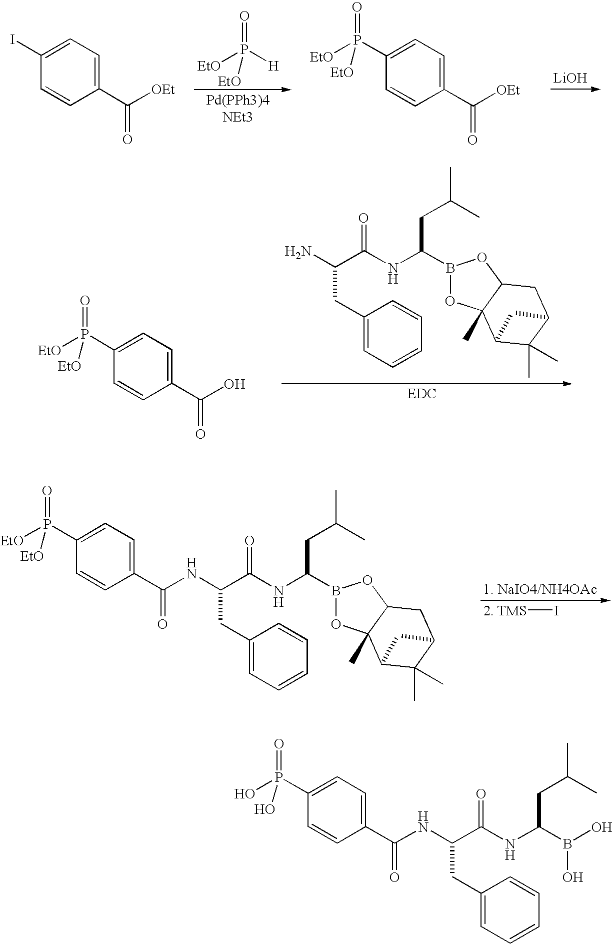

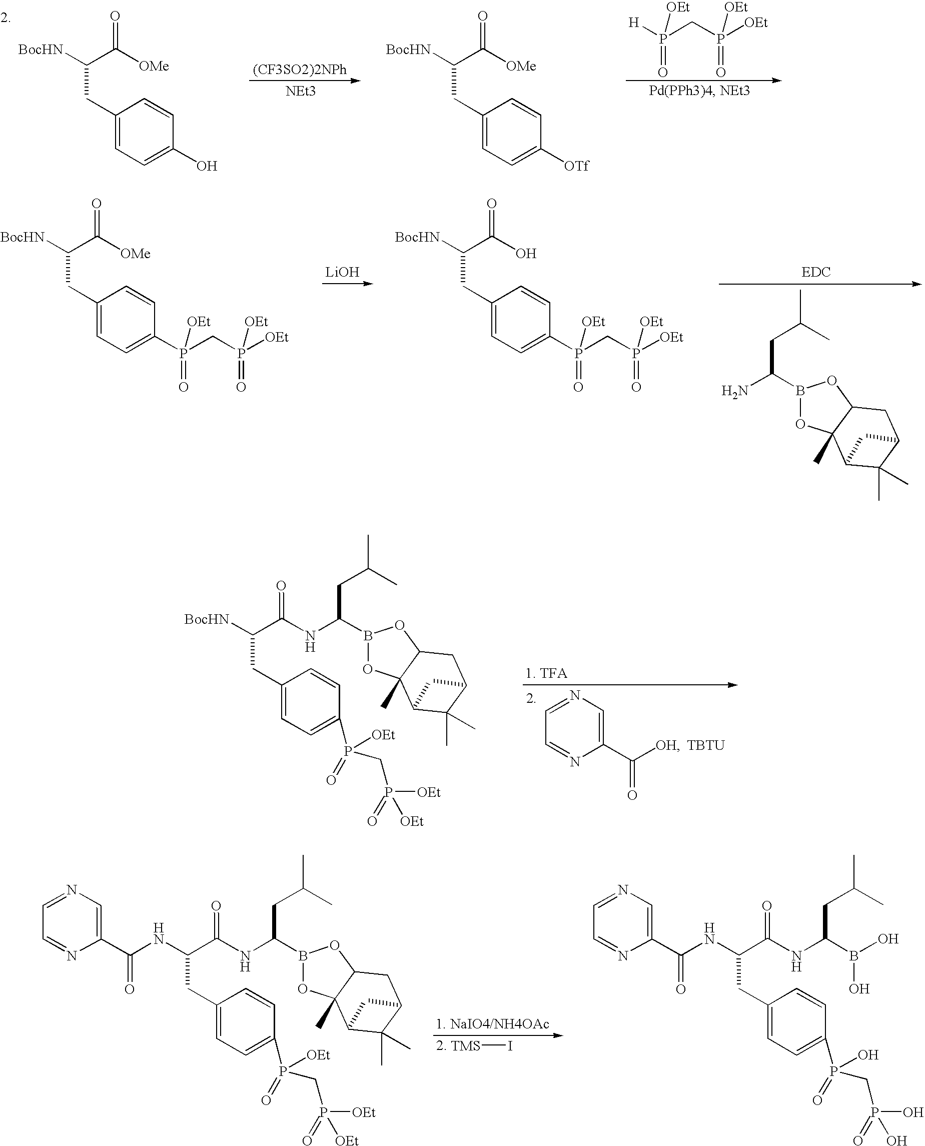

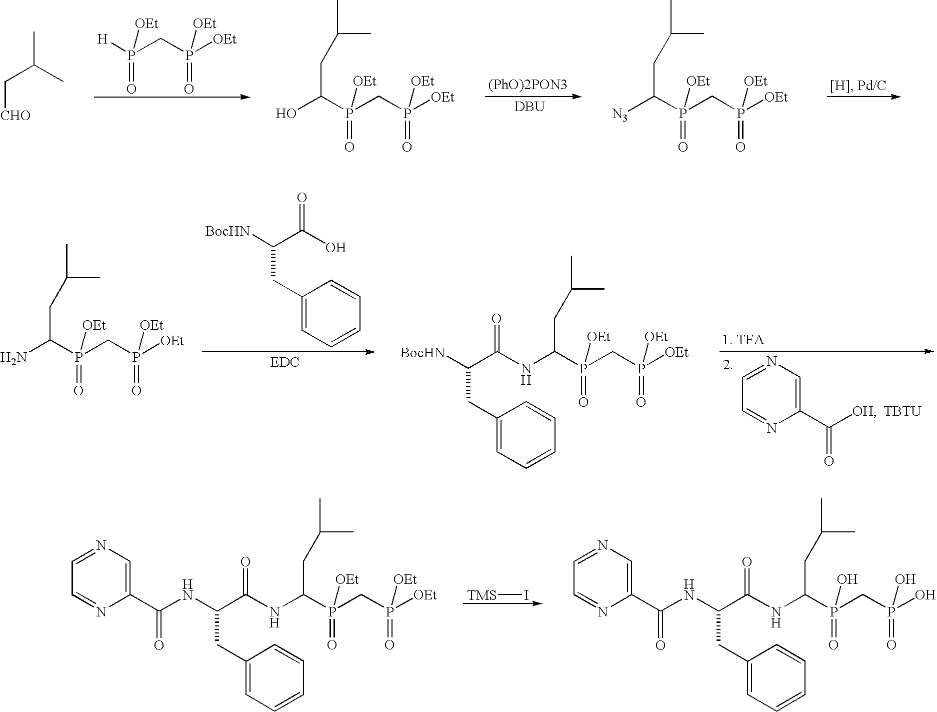

Solid Phase Synthesis and Combinatorial Libraries of Novel Peptide Compounds

It will be appreciated that, in addition to preparing the inventive compounds using traditional solution phase techniques, the present invention contemplates the preparation of compounds and libraries of compounds using solid phase techniques. Thus, the desired components may be modified so that they may be attached to the solid support. The use of a solid support bound component enables the use of more rapid split and pool techniques to generate larger libraries (e.g., greater than 10,000 members) more easily. It will be appreciated that solid phase parallel synthesis techniques also can be utilized, such as those described in U.S. Pat. Nos. 5,712,171 and 5,736,412; incorporated herein by reference.

A solid support, for the purposes of this invention, is defined as an insoluble material to which compounds are attached during a synthesis sequence. The use of a solid support is advantageous for the synthesis of libraries because the isolation of support-bound reaction products can be accomplished simply by washing away reagents from the support-bound material and therefore the reaction can be driven to completion by the use of excess reagents. Additionally, the use of a solid support also enables the use of specific encoding techniques to “track” the identity of the inventive compounds in the library. A solid support can be any material which is an insoluble matrix and can have a rigid or semi-rigid surface. Exemplary solid supports include, but are not limited to, pellets, disks, capillaries, hollow fibers, needles, pins, solid fibers, cellulose beads, pore-glass beads, silica gels, polystyrene beads optionally cross-linked with divinylbenzene, grafted co-poly beads, poly-acrylamide beads, latex beads, dimethylacrylamide beads optionally crosslinked with N-N′-bis-acryloylethylenediamine, and glass particles coated with a hydrophobic polymer. One of ordinary skill in the art will realize that the choice of particular solid support will be limited by the comparability of the support with the reaction chemistry being utilized. An exemplary solid support is a Tentagel amino resin, a composite of 1) a polystyrene bead crosslinked with divinylbenzene and 2) PEG (polyethylene glycol), is employed for use in the present invention. Tentagel is a particularly useful solid support because it provides a versatile support for use in on-bead or off-bead assays, and it also undergoes excellent swelling in solvents ranging from toluene to water.

Specific compounds may be attached directly to the solid support or may be attached to the solid support through a linking reagent. Direct attachment to the solid support may be useful if it is desired not to detach the library member from the solid support. For example, for direct on-bead analysis of biological/pharmacological activitiy or analysis of the compound structure, a stronger interaction between the library member and the solid support may be desirable. Alternatively, the use of a linking reagent may be useful if more facile cleavage of the inventive library members from the solid support is desired.

Furthermore, any linking reagent used in the present invention may comprise a single linking molecule, or alternatively may comprise a linking molecule and one or more spacer molecules. A spacer molecule is particularly useful when the particular reaction conditions require that the linking molecule be separated from the library member, or if additional distance between the solid support/linking unit and the library member is desired. In one particularly preferred embodiment, photocleavable linkers are employed to attach the solid phase resin to the component. Photocleavable linkers are advantageous because of the ability to use these linkers in in vivo screening strategies. Once the compound is released from the solid support via photocleavage, the compound is able to enter the cell. Exemplary photocleavable linkers include, but are not limited to ortho-Nitrobenzyl photolinkers and dithiane protected benzoin photolinkers. One of ordinary skill in the art will realize that the method of the present invention is not limited to the use of photocleavable linkers; rather other linkers may be employed, preferably those that are capable of delivering the desired compounds in vivo.

Thus, the synthesis of libraries of compounds can be performed using established combinatorial methods for solution phase, solid phase, or a combination of solution phase and solid phase synthesis techniques. The synthesis of combinatorial libraries is well known in the art and has been reviewed (see, e.g., “Combinatorial Chemistry”, Chemical and Engineering News, Feb. 24, 1997, p. 43; Thompson, L. A., Ellman, J. A., Chem. Rev. 1996, 96, 555, incorporated herein by reference.) One of ordinary skill in the art will realize that the choice of method will depend upon the specific number of compounds to be synthesized, the specific reaction chemistry, and the availability of specific instrumentation, such as robotic instrumentation for the preparation and analysis of the inventive libraries. In particularly preferred embodiments, the reactions to be performed on the inventive scaffolds to generate the libraries are selected for their ability to proceed in high yield, and in a stereoselective fashion, if applicable.

In one embodiment of the present invention, libraries are generated using a solution phase technique. Traditional advantages of solution phase techniques for the synthesis of combinatorial libraries include the availability of a much wider range of organic reactions, and the relative ease with which products can be characterized. In a preferred embodiment, for the generation of a solution phase combinatorial library, a parallel synthesis technique is utilized, in which all of the products are assembled separately in their own reaction vessels. In a particularly preferred parallel synthesis procedure, a microtitre plate containing n rows and m columns of tiny wells which are capable of holding a few milliliters of the solvent in which the reaction will occur, is utilized. It is possible to then use n variants of reactant A, and m variants of reactant B, to obtain n×m variants, in n×m wells. One of ordinary skill in the art will realize that this particular procedure is most useful when smaller libraries are desired, and the specific wells can provide a ready means to identify the library members in a particular well.

In another embodiment of the present invention, a solid phase synthesis technique is utilized, in which the desired scaffold structures are attached to the solid phase directly or though a linking unit, as discussed above. Advantages of solid phase techniques include the ability to more easily conduct multi-step reactions and the ability to drive reactions to completion because excess reagents can be utilized and the unreacted reagent washed away. Perhaps one of the most significant advantages of solid phase synthesis is the ability to use a technique called “split and pool”, in addition to the parallel synthesis technique, develped by Furka. (Furka et al., Abstr. 14th Int Congr. Biochem., Prague, Czechoslovakia, 1988, 5, 47; Furka et al., Int. J. Pept. Protein Res. 1991, 37, 487; Sebestyen et al., Bioorg. Med. Chem. Lett., 1993, 3, 413) In this technique, a mixture of related compounds can be made in the same reaction vessel, thus substantially reducing the number of containers required for the synthesis of very large libraries, such as those containing as many as or more than one million library members. As an example, the solid support scaffolds can be divided into n vessels, where n represents the number species of reagent A to be reacted with the scaffold structures. After reaction, the contents from n vessels are combined and then split into m vessels, where m represents the number of species of reagent B to be reacted with the scaffold structures. This procedure is repeated until the desired number of reagents is reacted with the scaffold structures to yield the inventive library.

The use of solid phase techniques in the present invention may also include the use of a specific encoding technique. Specific encoding techniques have been reviewed by Czarnik. (Czarnik, A. W., Current Opinion in Chemical Biology, 1997, 1, 60) As used in the present invention, an encoding technique involves the use of a particular “identifiying agent” attached to the solid support, which enables the determination of the structure of a specific library member without reference to its spatial coordinates. One of ordinary skill in the art will also realize that if smaller solid phase libraries are generated in specific reaction wells, such as 96 well plates, or on plastic pins, the reaction history of these library members may also be identified by their spatial coordinates in the particular plate, and thus are spatially encoded. It is most preferred, however for large combinatorial libraries, to use an alternative encoding technique to record the specific reaction history.

Examples of alternative encoding techniques that can be utilized in the present invention include, but are not limited to, spatial encoding techniques, graphical encoding techniques, including the “tea bag” method, chemical encoding methods, and spectrophotometric encoding methods. Spatial encoding refers to recording a reaction's history based on its location. Graphical encoding techniques involve the coding of each synthesis platform to permit the generation of a relational database. Examples of preferred spectrophotometic encoding methods include the use of mass spectroscopy, fluorescence emission, and nuclear magnetic resonance spectroscopy. In a preferred embodiment, chemical encoding methods are utilized, which uses the structure of the reaction product to code for its identity. Decoding using this method can be performed on the solid phase or off of the solid phase. One of ordinary skill in the art will realize that the particular encoding method to be used in the present invention must be selected based upon the number of library members desired, and the reaction chemistry employed.

Subsequent characterization of the library members, or individual compounds, can be performed using standard analytical techniques, such as mass spectrometry, Nuclear Magnetic Resonance Spectroscopy, and gas chromatrograpy.

Once specific libraries of compounds have been prepared, specific assay techniques, such as those described herein, may be utilized to test the activity of the inventive compounds. In certain preferred embodiments, high throughput assay techniques are utilized.

### EXAMPLE 2

In vitro and In vivo Assays:

Compounds of the present invention may be evaluated in a variety of assays to determine or characterize their biological activities. For example, the compounds of the invention can be tested for their ability to bind to bone, to inhibit bone resorption or to otherwise improve the relative dynamics of bone homeostasis. The compounds can also be evaluated for their cytotoxic and growth inhibitory effects on tumor cells of interest. Furthermore, the compounds can be evaluated for their ability to act as inhibitors of cell adhesion.

A. Anti-Resorption Cell Assay (Rabbit Osteoclast):

Femurs, tibias, and scapulas are isolated from 3-4 day old New Zealand white rabbits (Millbrook Farms, Amherst, Mass.). Bones are chopped and minced in a-MEM (Gibco-BRL) containing 0.55 g/L NaHCO₃, 10 mM HEPES (Gibco-BRL), 50 units/ml penicillin, and 0.05 mg/ml streptomycin, pH 7.1, Bone fragments are allowed to settle by gravitation, supernatant was collected and centrifuged at 400 RPM (Beckman GS-6KR) for two minutes, and the cell pellet is resuspended in the same medium supplemented with 10% HIFBS (Hyclone). For prebinding experiments, 0.75 ml of cell suspension is added to wells containing sperm whale dentine discs preincubated for 2 hours with 0.75 ml culture medium containing a 2× concentration of test compound. Alternatively, 0.75 ml of cell suspension is added to each well containing dentine slices preincubated with 0.75 ml culture medium alone and test compound is added after the adhesion phase. Sperm whale dentine was cut as 1 mm×6 mm circular discs. The adhesion phase was carried out for 30 minutes at 37° C. and 5% CO₂ and then the medium and non-adherent cells and debris were removed by aspiration. Fresh culture medium containing serially diluted test compounds is added and cells were incubated on dentine for 24 hours at 37° C. and 5% CO₂. After the resorption phase, dentine slices are soaked for 30 seconds in 0.5% sodium hypochlorite, wiped clean of adherent cells, and then stained for 30-45 seconds with 1% toluidine blue. Resorption is measured using reflective light microscopy and automated image analysis. The resorbed area is measured on the entire 6 mm disc. Remaining cells in the 24-well plates are stained for tartrate resistant acid phosphatase (TRAP) and also assessed visually for the presence of fibroblasts. Experiments are carried out containing triplicate samples for each concentration of compound tested with five untreated control samples per plate. IC₅₀ values are calculated based on the % resorption in the presence of compound relative to vehicle alone treated control samples. Data are calculated from at least three independent experiments each containing triplicate samples.

Generally speaking, in this assay, IC₅₀ values below about 10 _M are of particular interest, while scores below 500 nM or below are preferred, and scores below about 100 nM are particularly preferred.

B. Hydroxyapatite Assay:

Hydroxyapatite is the principal mineral component of bone. Hydroxyapatite adsorption chromatography is used as an assay to evaluate the bone-targeting potential of both individual bone-targeting moieties (“monomers”) and of pharmaceuticals incorporating bone-targeting groups.

Method: The rentention time of a test compound is measured using a linear gradient from 10 mM sodium phosphate, 0.15 N NaCl, pH=6.8 to 500 mM sodium phosphate, 0.15 N NaCl, pH=−6.8 on a TSK-Gel HA 1000 high pressure liquid chromatography column (7.5 mm×75 mm). The rentention time of the compound is expressed in terms of K=(retention time-void time)/void. This K value is corrected using two reference compounds to correct from inter-column and inter-system variation to obtain a K′ value.

Reference Compounds: K′ values were determined for known bone targeted compounds, the bisphosphonate, alendronate and tetracycline. Alendronate gave a K′ value of 3.7 and tetracycline gave a K′ value of 2.0.

C. Hypercalcemic Mouse Model for Testing In Vivo Anti Resorptive Activity

A murine hypercalcemia model for determining the efficacy of Src kinase inhibitors was developed. This model exploits the intrinsic effects of PTH (1-34) to stimulate the resorptive activity of osteoclasts in vivo. Briefly, compounds are each injected into mice subcutaneously, once or twice per day for five consecutive days. On the third day of test compound treatments, PTH administration begins. PTH (20 μg/kg) is given four times per day, subcutaneously, until the end of the study. Control animals receive PTH but do not receive test compounds. Blood samples are collected from the animals to obtain baseline (pre-PTH treatment), 48 hour and 72 hour (after initiation of PTH treatment) serum samples. The serum samples are analyzed for calcium concentration using the quantitative colorimetric assay reagent Arsenazo III (Sigma). Calcium serum levels for treated groups are compared to calcium serum levels of control groups and a percentage of inhibition of hypercalcemia is calculated for each time point. When a compound is effective in inhibiting the activity of osteoclasts, observed serum calcium concentrations are lower than those in animals that receive only PTH in the absence of test compound.

D. Cytoxicity and Inhibition of Tumor Growth:

Certain compounds of this invention have also demonstrated cytotoxic and antitumor activity and thus may be useful in the treatment of cancer and other cell proliferative diseases. Compounds are assayed for anti-tumor activity using in vivo and in vitro assays which are well known to those skilled in the art. Generally, initial screens of compounds to identify candidates for anti-cancer drugs are performed in cellular in vitro assays. Compounds identified as having anti-cell proliferative activity can then be subsequently assayed in whole organisms for anti-tumor activity and toxicity. The initial screens are preferably cellular assays which can be performed rapidly and cost-effectively relative to assays that use whole organisms. For purposes of the present invention, the term “anti-proliferative compound” is used to mean compounds having the ability to impede or stop cells from progressing through the cell cycle and dividing. For purposes of the present invention, the terms “anti-tumor” and “anti-cancer” activity are used interchangeably.

Methods for determining cell proliferation are well known and can be used to identify compounds with anti-proliferative activity. In general, cell proliferation and cell viability assays are designed to provide a detectable signal when cells are metabolically active. Compounds are tested for anti-cell proliferation activity by assaying for a decrease in metabolic activity. Commonly used methods for determining cell viability depend upon, for example, membrane integrity (e.g. trypan blue exclusion) or incorporation of nucleotides during cell proliferation (e.g. BrdU or ³H-thymidine).

Preferred methods of assaying cell proliferation utilize compounds that are converted into a detectable compound during cell proliferation. Particularly preferred compounds are tetrazolium salts and include without limitation MTT (3-(4,5-dimethylthiazol-2-yl)-2,5-diphenyltetrazolium bromide; Sigma-Aldrich, St. Louis, Mo.), MTS (3-(4,5-dimethylthiazol-2-yl)-5-(3-carboxymethoxyphenyl)-2-(4-sulfophenyl)-2H-tetrazolium), XTT (2,3-bis(2-Methoxy-4-nitro-5-sulfophenyl)-2H-tetrazolium-5-carboxanilide), INT, NBT, and NTV (Bernas et al. Biochim Biophys Acta 1451(1):73-81, 1999). Preferred assays utilizing tetrazolium salts detect cell proliferation by detecting the product of the enzymatic conversion of the tetrazolium salts into blue formazan derivatives, which are readily detected by spectroscopic methods (Mosman. J. Immunol. Methods. 65:55-63, 1983).

Generally, preferred methods for assaying cell proliferation involve incubating cells in a desired growth medium with and without the compounds to be tested. Growth conditions for various prokaryotic and eukaryotic cells are well-known to those of ordinary skill in the art (Ausubel et al. Current Protocols in Molecular Biology. Wiley and Sons. 1999; Bonifacino et al. Current Protocols in Cell Biology. Wiley and Sons. 1999 both incorporated herein by reference). To detect cell proliferation, the tetrazolium salts are added to the incubated cultured cells to allow enzymatic conversion to the detectable product by active cells. Cells are processed, and the optical density of the cells is determined to measure the amount of formazan derivatives. Furthermore, commercially available kits, including reagents and protocols, are availabe for examples, from Promega Corporation (Madison, Wis.), Sigma-Aldrich (St. Louis, Mo.), and Trevigen (Gaithersburg, Md.).

Any cultured cell line may be used to screen compounds for antiproliferative activity. In certain embodiments of the invention cell lines utilized include, but are not limited to, Exemplary cell lines utilized for the determination of the ability of inventive compounds to inhibit cellular proliferation include, but are not limited to COLO 205 (colon cancer), DLD-1 (colon cancer), HCT-15 (colon cancer), HT29 (colon cancer), HEP G2 (Hepatoma), K-562 (Leukemia), A549 (Lung), NCl —H249 (Lung), MCF7 (Mammary), MDA-MB-231 (Mammary), SAOS-2 (Osteosarcoma), OVCAR-3 (Ovarian), PANC-1 (Pancreas), DU-145 (Prostate), PC-3 (Prostate), ACHN (Renal), CAKI-1 (Renal), MG-63 (Sarcoma).

Preferably, the cell line is a mammalian, but is not limited to mammalian cells since lower order eukaryotic cells such as yeast may also be used to screen compounds. Preferred mammalian cell lines are derived from humans, rats, mice, rabbits, monkeys, hamsters, and guinea pigs since cells lines from these organisms are well-studied and characterized. However, the present invention does not limit the use of mammalians cells lines to only the ones listed.

Suitable mammalian cell lines are often derived from tumors. For example, the following tumor cell-types may be sources of cells for culturing cells: melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Non-limiting examples of mammalian cells lines that have been widely used by researchers include HeLa, NIH/3T3, HT1080, CHO, COS-1, 293T, WI-38, and CV-1/EBNA-1.

Other in vitro cellular assays may be used which rely upon a reporter gene to detect metabolically active cells. Non-limiting examples of reporter gene expression systems include green fluorescent protein (GFP), and luciferase. As an example of the use of GFP to screen for potential antitumor drugs, Sandman et al. (Chem Biol. 6:541-51; incorporated herein by reference) used HeLa cells containing an inducible variant of GFP to detect compounds that inhibited expression of the GFP, and thus inhibited cell proliferation.

Compounds identified by in vitro cellular assays as having anti-cell proliferation activity are then tested for anti-tumor activity in whole organisms. Preferably, the organisms are mammalian. Well-characterized mammalians systems for studying cancer include rodents such as rats and mice. Typically, a tumor of interest is transplanted into a mouse having a reduced ability to mount an immune response to the tumor to reduce the likelihood of rejection. Such mice include for example, nude mice (athymic) and SCID (severe combined immunodeficiency) mice. Other transgenic mice such as oncogene containing mice may be used in the present assays (see for example U.S. Pat. No. 4,736,866 and U.S. Pat. No. 5,175,383). For a review and discussion on the use of rodent models for antitumor drug testing see Kerbel (Cancer Metastasis Rev. 17:301-304, 1998-99).

In general, the tumors of interest are implanted in a test organism preferably subcutaneously. The organism containing the tumor is treated with doses of candidate anti-tumor compounds. The size of the tumor is periodically measured to determine the effects of the test compound on the tumor. Some tumor types are implanted at sites other than subcutaneous sites (e.g., at intrapertoneal sites) and survival is the measured endpoint. Parameters to be assayed with routine screening include different tumor models, various tumor and drug routes, and doses amounts and schedule. For a review of the use of mice in detecting antitumor compounds see Corbett et al. (Invest New Drugs. 15:207-218, 1997; incorporated herein by reference).

E. Assays for Inhibition of the ATP Ubiquitin-Dependent Degradative Process:

One approach to testing compounds for their ability to inhibit the ATP-ubiquitin-dependent degradative process is to measure proteolysis in cultured cells (Rock, et al., Cell 78:761 (1994)). For example, the degradation of long-lived intracellular proteins is measured in mouse C2C12 myoblast cells. Cells are incubated with ³⁵S-methionine for 48 hours to label long-lived proteins and then chased for 2 hours with medium containing unlabeled methionine. After the chase period, the cells are incubated for 4 hours in the presence or absence of the test compound. The amount of protein degradation in the cell is measured by quantitating the trichloroacetic acid soluble radioactivity released from the pre-labeled proteins into the growth medium (an indicator of intracellular proteolysis).

For example, C2C12 cells (a mouse myoblast line) are labelled for 48 hrs with ³⁵S-methionine. The cells are then washed and preincubated for 2 hrs in the same media supplemented with 2 mM unlabelled methionine. The media is removed and replaced with a fresh aliquot of the preincubation media containing 50% serum, and a concentration of the compound to be tested. The media is then removed and made up to 10% TCA and centrifuged. The TCA soluble radioactivity is counted. Inhibition of proteolysis is calculated as the percent decrease in TCA soluble radioactivity. From this data, an EC₅₀ for each compound is calculated.

F. Assay to Determine the Ability of Compounds to Inhibit NF-kB

This assay is performed as described (Palombella, et al. Cell, 78.773-785 (1994)). MG63 osteosarcoma cells are stimulated by treatment with TNF-a for the designated times. Whole cell extracts are prepared and analyzed by electrophoretic mobility shift assay using the PRDII probe from the human IFN-b gene promoter.

G. Assay to Determine Inhibition of Expression of Cell Adhesion Molecules on HUVE Cells

HUVECs in microtiter plates are exposed to the indicated concentrations of inhibitor for 1 hour, prior to the addition of 100 U/mL TNF-a. Cell surface binding assays are performed at 4° C., using saturating concentrations of monoclonal antibodies specific for the cell adhesion molecules (Becton Dickenson) and fluorescent-conjugated F(ab′)₂ goat anti-murine IgG (Caltag Labs, San Francisco, Calif.). Fluorescent immunoassays for E-selectin and I-CAM are performed at 4 hours, those for V-CAM at 16 hours.

H. Assay to Determine the Ability of Compounds to Inhibit NS3 Serine Protease:

Insofar as inventive compounds are able to inhibit NS3 serine protease, they are of evident clinical utility for the treatment of viral diseases, including HCV. Exemplary procedures are described in U.S. Pat. No. 6,265,380, the entire contents of which is hereby incorporated by reference.

### EXAMPLE 3

Pharmaceutical Formulations:

The following formulations exemplify typical pharmaceutical compositions in dosage unit form suitable for systemic or topical administration to warm-blooded animals in accordance with the present invention.

“Active ingredient” (A.I.), as used herein, relates to a compound of formula (I) and all classes and subsets as described herein, a pharmaceutically acceptable derivative thereof, or a stereochemically isomeric form thereof.

A. Oral Solutions:

9 g of methyl 4-hydroxybenzoate and 1 g of propyl 4-hydroxybenzoate are dissolved in 4 l of bioling purified water. In 3 l of this solution are dissolved first 10 g of 2,3-dihydroxybutanedioic acid and thereafter 20 grams of the active ingredient. The latter solution is combined with the remaining part of the former solution and 12 l of 1,2,3-propanetriol and 3 l of sorbitol 70% solution are added thereto. 40 g of sodium saccharin are dissolved in 0.5 l of water and 2 ml of raspberry and 2 ml of gooseberry essence are added. The latter solution is combined with the former, water is added q.s. to a volume of 20 l providing an oral solution comprising 5 mg of the active ingredient per teaspoonfull (5 ml). The resulting solution is filled in suitable containers.

B. Capsules:

20 grams of the active ingredient, 6 g sodium lauryl sulfate, 56 g starch, 56 g lactose, 0.8 g colloidal silicon dioxide, and 1.2 g magnesium stearate are vigorously stirred together. The resulting mixture is subsequently filled into 1000 suitable hardened gelatin capsules, each comprising 20 mg of the active ingredient.

C. Film-Coated Tablets:

Preparation of tablet core: A mixture of 100 g of the active ingredient, 570 g lactose and 200 g starch is mixed well and thereafter humidified with a solution of 5 g sodium dodecyl sulfate and 10 g polyvinyl pyrrolidone in about 200 ml of water. The wet powder mixture is sieved, dried and sieved again. Then there are added 100 g microcrystalline cellulose and 15 g hydrogenated vegatable oil. The whole is mixed well and compressed into tablets, giving 10,000 tablets, each comprising 10 mg of the active ingredient.

Coating: To a solution of 10 g methyl cellulose in 75 ml of denatrurated ethanol is added a solution of 5 g of ethyl cellulose in 150 ml of dichloromethane. Then there are added 75 ml of dichloromethane and 2.0 ml 1,2,3-propanetriol. 10 g of polyethylene glycol is molten and dissolved in 75 ml of dichloromethane. The latter solution is added to the former and then 2.5 g of magnesium octadecoanoate, 5 g polyvinylpyrrolidone and 30 ml of concentrated color suspension is added and the mixture is homogenated. The tablet cores are coated with the mixture in a coating apparatus.

D. Injectable Solution:

1.8 g methyl 4-hydroxybenzoate and 0.2 g propyl 4-hydroxybenzoate were dissolved in about 0.5 l of boiling water for injection. After cooling to about 50 C., 4 g lactic acid. 0.05 g propylene glycol, and 4 grams of the active ingredient were added while stirring. The solution was then cooled to room temperature and supplemented with water for injection q.s. ad 11 volume, giving a solution of 4 mg/ml of active ingredient. The solution was sterilized by filtration and filled in sterile containers.

1. A compound having the structure (I): 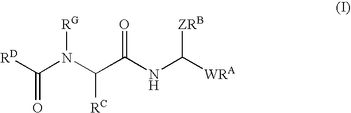
or a pharmaceutically acceptable derivative thereof,
wherein R^{A} and R^{B} are each independently hydrogen, COOH, B(OH)₂, a phosphorus-containing moiety, or an ester of any of the foregoing, or an aliphatic, heteroaliphatic, aryl or heteroaryl moiety;
Z and W are each independently a covalent bond or an aliphatic, heteroaliphatic, aryl, or heteroaryl linker of 1-10 carbon atoms;
R^{C} is an aliphatic, heteroaliphatic, aryl, heteroaryl, aliphatic(aryl), aliphatic(heteroaryl), heteroaliphatic(aryl), or heteroaliphatic(heteroaryl) moiety;
R^{D} is an aryl or heteroaryl moiety;
R^{G} is hydrogen or an alkyl group;
wherein in each of the foregoing groups Z, W, R^{C}, R^{D}, and R^{G} each alkyl, aliphatic, heteroaliphatic, alkylaryl, or alkylheteroaryl moiety may independently be branched or unbranched, cyclic or acyclic or substituted or unsubstituted, and may contain one or more electronically unsaturated bonds, and each aryl and heteroaryl moiety may independently be substituted or unsubstituted; and
at least one of R^{A}, R^{B}, R^{C} or R^{D} comprises or is substituted with a phosphorus-containing moiety, with the proviso that:
(i) if R^{A} is the only phosphorus-containing moiety; if R^{D} is a phenyl moiety substituted with a substituted or unsubstituted phenoxy or naphthyloxy moiety; and if -ZR^{B} is a phenyl, benzyl or C₁₋₆ alkyl moiety substituted with an amidino, guanidino, isothioureido or amino moiety, then R^{A} is not —P(═O)(OR^{Z})(R^{X}), where R^{Z} is C₁₋₆ perfluoroalkyl, phenyl or substituted phenyl and R^{X} is C₁₋₆ perfluoroalkoxy, phenoxy, halogen, C₁₋₆ alkoxy or substituted phenoxy; and
(ii) if R^{D} is the only phosphorus-containing moiety and is a quinoline moiety substituted with —PO₃H; then R^{C} is not an alkyl, aryl, or aralkyl moiety substituted with nitro or amino.

2. The compound of claim 1, wherein R^{C} is a benzyl moiety optionally substituted with one or more occurrences of R^{C} and R^{E} and the compound has the structure: 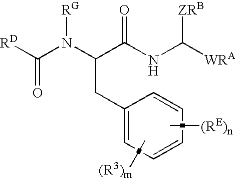
wherein each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6;
one or more of R^{A}, R^{B}, R^{D} or R^{E} comprises or is substituted with a phosphorus-containing moiety;
n is 0-3; and
m is 0-3, and the sum of n+m is an integer from 0-5.

3. The compound of claim 1, wherein ZR^{B} is —CH₂CH(CH₃)(CH₃) and the compound has the structure: 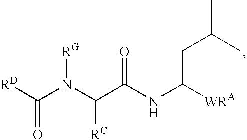
wherein at least one of R^{A}, R^{C} or R^{D} comprises or is substituted with a phosphorus-containing moiety.

4. The compound of claim 1, wherein R^{D} is an aryl moiety optionally substituted with one or more occurrences of R^{F} and R³, and the compound has the structure: 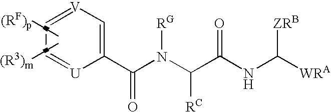
wherein each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6;
at least one of R^{A}, R^{B} or R^{F} comprises or is substituted with phosphorus-containing moiety;
V and U are each independently CR³ or N;
p is 0-3; and
m is 0-3, and the sum of m+p is an integer from 0-5.

5. The compound of claim 1, wherein WR^{A} is B(OH)₂ and the compound has the structure: 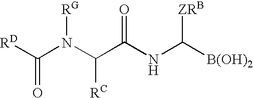
wherein at least one of R^{B}, R^{C} or R^{D} comprises or is substituted with a phosphorus-containing moiety.

6. The compound of claim 1, wherein R^{D} is an aryl moiety optionally substituted with one or more occurrences of R^{F} and R³, and R^{C} is a benzyl moiety optionally substituted with one or more occurrences of R^{E} and R³, and the compound has the structure: 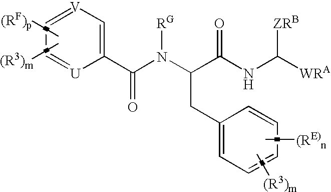
wherein each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted alkyl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6;
at least one of R^{A}, R^{B}, R^{E} or R^{F} comprises or is substituted with a phosphorus-containing moiety;
V and U are each independently N or CR³;
n is 0-3;
p is 0-3; and
m is 0-3, and the sum of m+p or n+m is an integer from 0-5.

7. The compound of claim 1, wherein R^{D} is an aryl moiety optionally substituted with one or more occurrences of R^{F} and R³, and R^{C} is a benzyl moiety optionally substituted with one or more occurrences of R^{E} and R³, ZR^{B} is isobutyl, and the compound has the structure: 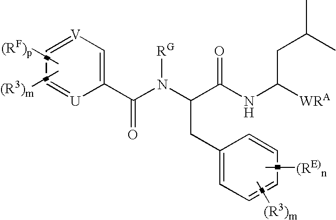
wherein each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6;
at least one of R^{A}, R^{E} or R^{F} comprises or is substituted with a phosphorus-containing moiety;
V and U are each independently N or CR³;
n is 0-3;
p is 0-3; and
m is 0-3, and the sum of m+p or n+m is an integer from 0-5.

8. The compound of any one of claims 1-7, wherein only one of R^{A}, R^{B}, R^{C}, R^{D}, R^{E} or R^{F} comprises or is substituted with a phosphorus-containing moiety.

9. The compound of any one of claims 1-7, wherein two or more of R^{A}, R^{B}, R^{C}, R^{D}, R^{E} or R^{F} comprise or are substituted with a phosphorus-containing moiety.

10. The compound of any one of claims 1-7, wherein one or more occurrences of R^{A}, R^{B}, R^{C}, R^{D}, R^{E} or R^{F} comprises or is substituted with a phosphorus-containing moiety of Series I: 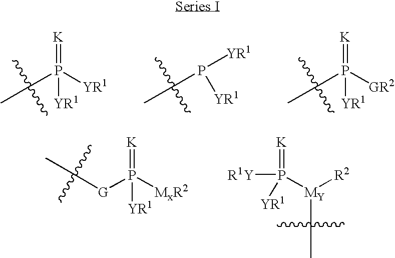
wherein each occurrence of K is independently O or S;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of R² is independently R¹, —PK(YR¹)(YR¹), —SO₂(YR¹) or —C(O)(YR¹);
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6; and
each occurrence of M_{Y} is independently a methine group or a lower (i.e., of 1-6 carbons linked to one another through carbon-carbon bonds) alkyl moiety which contains a methine group and optionally may be further substituted.

11. The compound of any one of claims 1-7, wherein one or more occurrences of R^{A}, R^{B}, R^{C}, R^{D}, R³ or R^{F} comprises or is substituted with a phosphorus-containing moiety of Series Ia: 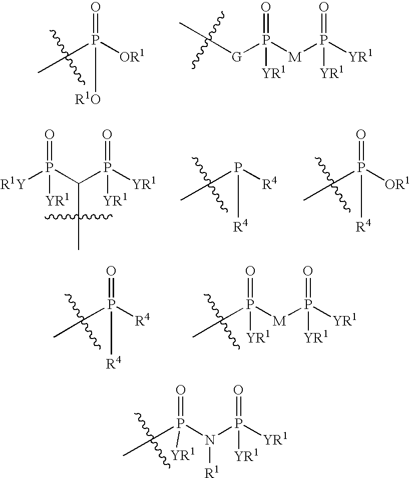
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6; and
each occurrence of R⁴ is independently an aliphatic, heteroaliphatic, aryl, or heteroaryl moiety.

12. The compound of claim 11, wherein Y is O.

13. The compound of any one of claims 1-7, wherein one or more occurrences of R^{A}, R^{B}, R^{C}, R^{D}, R^{E} or R^{F} comprises or is substituted with a phosphorus-containing moiety of Series Ib: 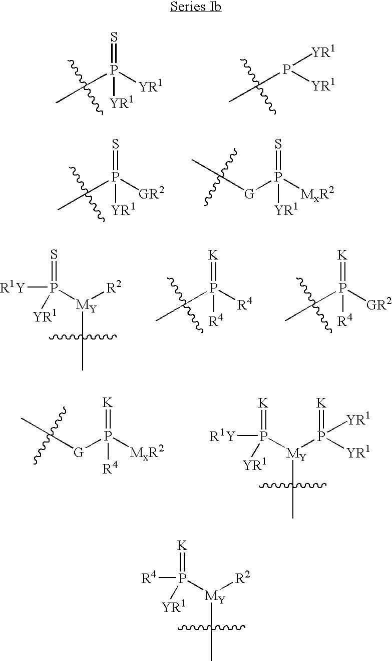
wherein each occurrence of K is independently O or S;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of R² is independently R¹, —PK(YR¹)(YR¹), —SO₂(YR¹) or —C(O)(YR¹);
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6; and
each occurrence of M_{Y} is independently a methine group or a lower alkyl moiety which contains a methine group and optionally may be further substituted; and
each occurrence of R⁴ is independently an aliphatic, heteroaliphatic, aryl, or heteroaryl moiety.

14. The compound of claim 13, wherein Y is O.

15. The compound of any one of claims 1, 2, 3, 4, 6 or 7 wherein W is a covalent bond and R^{A} is B(OH)₂.

16. The compound of any one of claims 1, 2, 3, 4, 6 or 7, wherein W is a lower alkyl moiety and R^{A} is a phosphorus-containing moiety.

17. The compound of claims 1, 2, 4, 5, or 6, wherein ZR^{B} is a linear or branched, cyclic or acyclic, substituted or unsubstituted alkyl moiety.

18. The compound of any one of claims 1, 2, 4, 5, or 6, wherein ZR^{B} is, —CH₂(cyclopentyl), —CH₂(cyclohexyl), —CH₃, —CH₂CH₃, —CH₂CH₂CH₃, —CH₂CH₂CH₂CH₃, or —CH₂CH(CH₃)(CH₃).

19. The compound of any one of claims 1, 2, 4, 5, or 6, wherein Z is a lower alkyl moiety and R^{B} is a phosphorus-containing moiety.

20. The compound of any one of claims 1, 3, 4 or 5, wherein R^{C} is a substituted or unsubstituted aryl, alkylaryl, heteroaryl, or alkylheteroaryl moiety.

21. The compound of any one of claims 1, 2, 3, or 5, wherein R^{D} is a substituted or unsubstituted aryl, alkylaryl, heteroaryl, or alkylheteroaryl moiety.

22. The compound of any one of claims 1-5, wherein either or both of R^{C} and R^{D} comprise any one of the following structures: 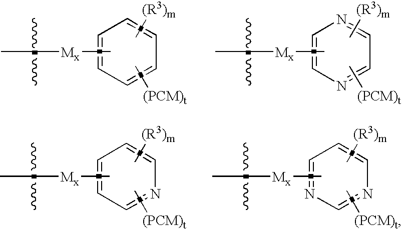
wherein each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6;
PCM is a phosphorus-containing moiety of Series I, Series Ia or Series Ib; and
m and t are each independently an integer from 0-3, and the sum of m+t is an integer from 0-5.

23. The compound of any one of claims 1-5, wherein either or both of R^{C} and R^{D} comprise any one of the following structures: 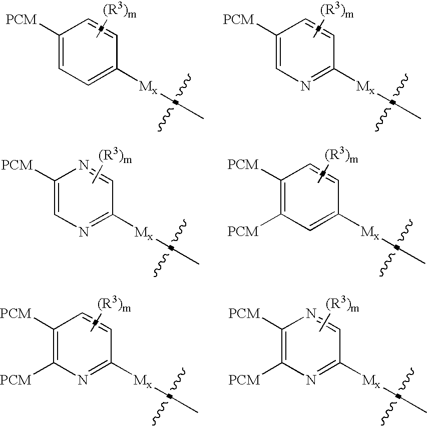
wherein each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6;
m is an integer from 0-3; and
PCM is a phosphorus-containing moiety of Series I, Series Ia or Series Ib.

24. The compound of any one of claims 1, 3, 4, or 5, wherein R^{C} comprises any one of the following structures: 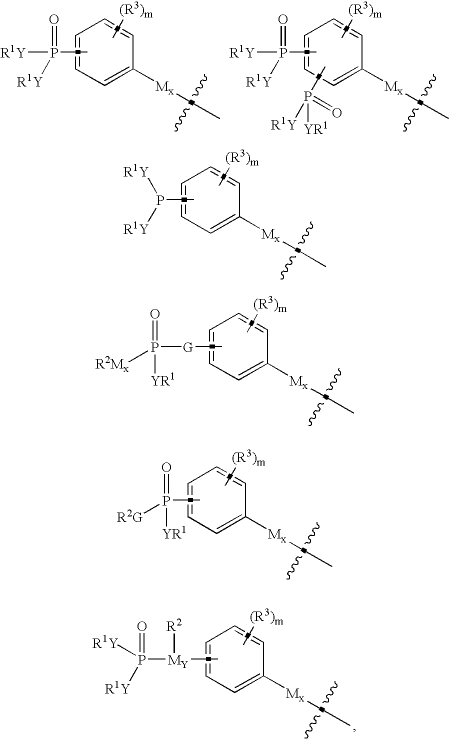
wherein each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6; and
m is an integer from 0-3.

25. The compound of any one of claims 1-3 or 5, wherein R^{D} comprises any one of the following structures: 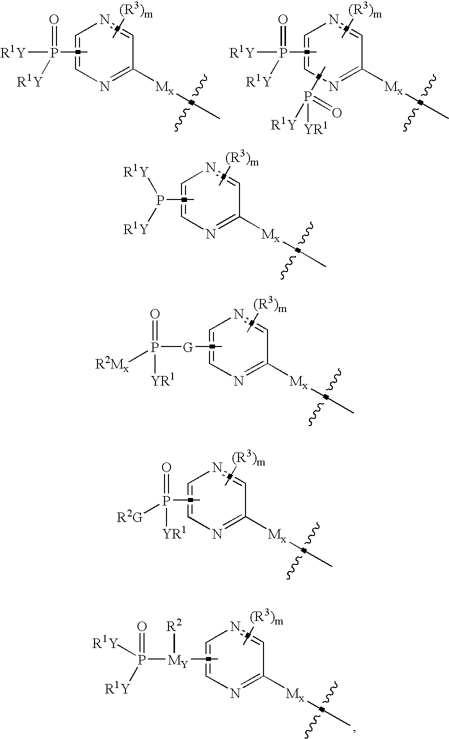
wherein each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6; and
m is an integer from 0-3.

26. The compound of any one of claims 1-7, wherein one or more occurrences of R^{A}, R^{B}, R^{C}, R^{D}, R^{E} or R^{F} comprise or are substituted with a phosphorus-containing moiety having the structure: 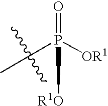
wherein each R¹ is independently H, alkyl, arylalkyl, aryl or a prodrug moiety.

27. The compound of any one of claims 1-7, wherein one or more occurrences of R^{A}, R^{B}, R^{C}, R^{D}, R^{E} or R^{F} comprise or are substituted with a phosphorus-containing moiety having the structure: 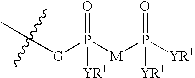
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H, or may also be a prodrug;
each occurrence of G is independently absent, or is —O—, —S—, —NR¹— or (M)_{X};
each occurrence of M is independently a substituted or unsubstituted methylene moiety, and any M-M′ moiety may be saturated or unsaturated; and
each occurrence of x is independently an integer from 0-6.

28. The compound of any one of claims 1-7, wherein one or more occurrences of R^{A}, R^{B}, R^{C}, R^{D}, R^{E} or R^{F} comprise or are substituted with a phosphorus-containing moiety having the structure: 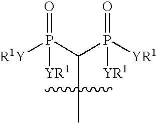
wherein each occurrence of Y is independently —O—, —S—, —NR¹—, or a chemical bond linking R¹ to P; and
each occurrence of R¹ is independently a substituted or unsubstituted aliphatic, heteroaliphatic, aryl or heteroaryl moiety, or, except in YR¹ moieties in which Y is a covalent bond, R¹ may also be H, or may also be a prodrug.

29. The compound of any one of claims 1-7, wherein one or more occurrences of R^{A}, R^{B}, R^{C}, R^{D}, R^{E} or R^{F} comprise or are substituted with a phosphorus-containing moiety having the structure: 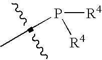
wherein each occurrence of R⁴ is independently alkyl, arylalkyl, aryl or a prodrug moiety;

30. The compound of any one of claims 1-7, wherein one or more occurrences of R^{A}, R^{B}, R^{C}, R^{D}, R^{E} or R^{F} comprise or are substituted with a phosphorus-containing moiety having the structure: 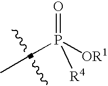
wherein R¹ is H, alkyl, arylalkyl or a prodrug moiety and R⁶ is alkyl, arylalkyl, aryl or a prodrug moiety.

31. The compound of any one of claims 1-7, wherein one or more occurrences of R^{A}, R^{B}, R^{C}, R^{D}, R^{E} or R^{F} comprise or are substituted with a phosphorus-containing moiety having the structure: 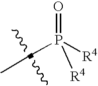
wherein each occurrence of R⁴ is independently alkyl, arylalkyl, aryl or a prodrug moiety.

32. The compound of any one of claims 1-7, wherein one or more occurrences of R^{A}, R^{B}, R^{C}, R^{D}, R^{E} or R^{F} comprise or are substituted with a phosphorus-containing moiety having the structure: 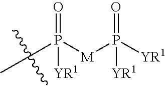
wherein each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H, or may also be a prodrug; and
each occurrence of M is independently a substituted or unsubstituted methylene moiety, and any M-M′ moiety may be saturated or unsaturated.

33. The compound of any one of claims 1-7, wherein one or more occurrences of R^{A}, R^{B}, R^{C}, R^{D}, R^{E} or R^{F} comprise or are substituted with a phosphorus-containing moiety having the structure: 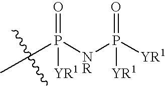
wherein each R¹ is independently H, alkyl, arylalkyl, aryl or a prodrug moiety and R is aliphatic, heteroaliphatic, aryl, or heteroaryl.

34. The compound of any one of claims 1-7, wherein one or more occurrences of R^{A}, R^{B}, R^{C}, R^{D}, R^{E} or R^{F} comprise or are substituted with a phosphorus-containing moiety having the structure:

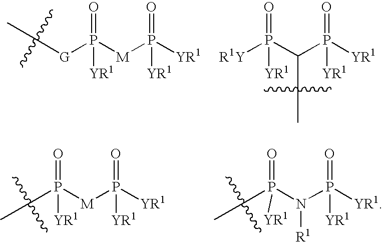

35. The compound of any one of claims 1-7, wherein R^{G} is hydrogen.

36. The compound of any one of claims 1-7, wherein R^{G} is lower alkyl.

37. The compound of any one of claims 2, 4, 6 or 7, wherein R³ is hydrogen.

38. The compound of any one of claims 1-6, wherein ZR^{B} and R^{C} may each independently be hydrogen, C₁₋₈ alkyl, C₃₋₁₀ alkyl, C₆₋₁₀ aryl or —CH₂R^{H}, wherein R^{H}, for each occurrence, is independently one of C₆₋₁₀ aryl, C₆₋₁₀aryl(C₁₋₆)alkyl, C₁₋₆alkyl(C₆₋₁₀)aryl, C₃₋₁₀cycloalkyl, C₁₋₈alkoxy, or C₁₋₈alkylthio, where the ring portion of any of the foregoing aryl, aralkyl, or alkaryl groups of ZR^{B}, R^{C} or R^{H} can optionally be substituted by one or two substituents independently selected from the group consisting of C₁₋₆alkyl, C₃₋₈cycloalkyl, C₁₋₆alkyl(C₃₋₈)cycloalkyl, C₂₋₈alkenyl, C₂₋₈alkynyl, cyano, amino, C₁₋₆alkylamino, di(C₁₋₆)alkylamino, benzylamino, dibenzylamino, nitro, carboxy, carbo(C₁₋₆)alkoxy, trifluoromethyl, halogen, C₁₋₆alkoxy, C₆₋₁₀aryl, C₆₋₁₀aryl(C₁₋₆)alkyl, C₆₋₁₀aryl(C₁₋₆)alkoxy, hydroxy, C₁₋₆alkylthio, C₁₋₆alkylsulfinyl, C₁₋₆alkylsulfonyl, C₆₋₁₀arylthio, C₆₋₁₀arylsulfinyl, C₆₋₁₀arylsulfonyl, C₆₋₁₀aryl, C₁₋₆alkyl(C₆₋₁₀)aryl, and halo(C₆₋₁₀)aryl.

39. A compound having the structure: 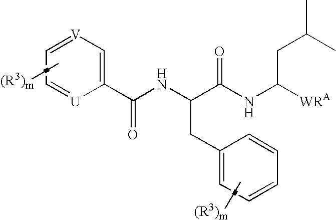
wherein W is —(CH₂)ₛ wherein s is an integer from 0-6;
V and U are each independently CR₃ or N;
R_{A} is a phosphorus-containing moiety of Series I, Ia or Ib;
wherein each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6; and
m is an integer from 0-3.

40. The compound of claim 39, wherein each occurrence of m is O and V and U are each N and the compound has the structure: 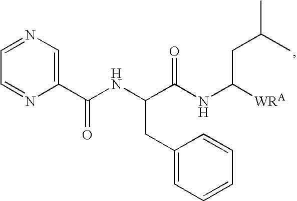
wherein R^{A} is a phosphorus-containing moiety of Series Ia: 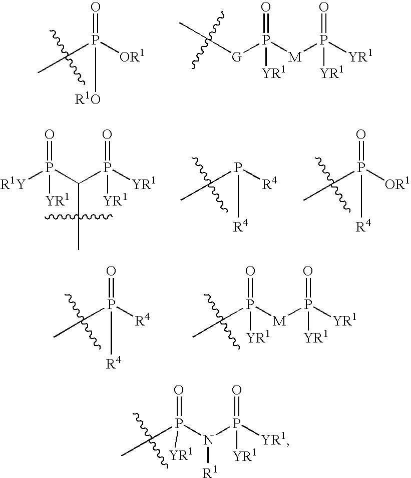
wherein each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6; and
each occurrence of R⁴ is independently alkyl, arylalkyl, aryl or a prodrug moiety.

41. A compound having the structure: 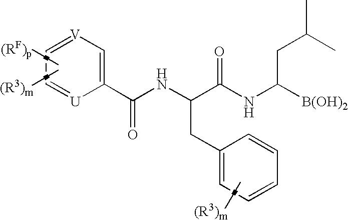
wherein V and U are each independently CR₃ or N;
R^{F} is a phosphorus-containing moiety of Series I, Ia or Ib;
wherein each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6;
p is 1 or 2; and
m is an integer from 0-3.

42. The compound of claim 41, wherein each occurrence of m is O and V and U are each N and the compound has the structure: 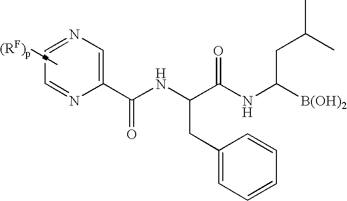
wherein R^{F} is a phosphorus-containing moiety of Series Ia: 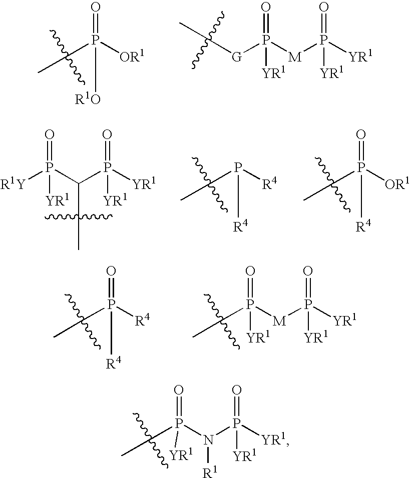
wherein each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6;
each occurrence of R⁴ is independently alkyl, arylalkyl, aryl or a prodrug moiety; and
p is 1 or 2.

43. A compound having the structure: 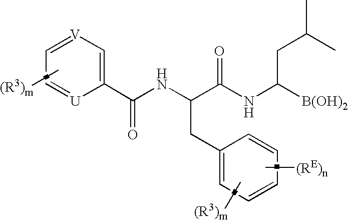
wherein V and U are each independently CR₃ or N;
R^{E} is a phosphorus-containing moiety of Series I, Ia or Ib;
each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6;
n is 1 or 2; and
m is an integer from 0-3.

44. The compound of claim 43, wherein each occurrence of m is O, V and U are each N and the compound has the structure: 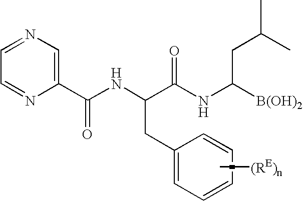
wherein R^{E} is a phosphorus-containing moiety of Series Ia: 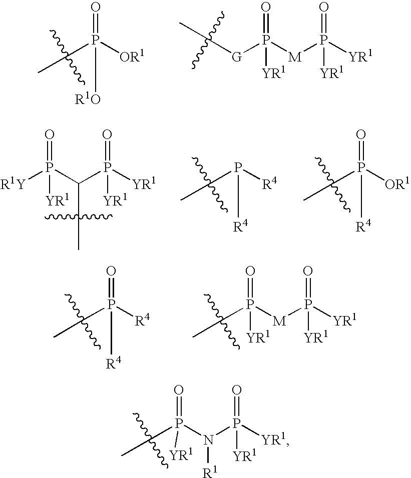
wherein each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6;
each occurrence of R⁴ is independently alkyl, arylalkyl, aryl or a prodrug moiety; and
n is 1 or 2.

45. A compound having the structure: 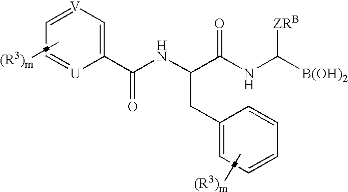
wherein Z is —(CH₂)ₛ wherein s is an integer from 0-6;
V and U are each independently CR₃ or N;
R^{B} is a phosphorus-containing moiety of Series I, Ia or Ib;
wherein each occurrence of R³ is independently halogen; -GR1; -GCO(YR1); -GSO2(YR1); cyano, nitro or azido;
each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6; and
m is an integer from 0-3.

46. The compound of claim 45, wherein each occurrence of m is O, V and U are each N, and the compound has the structure: 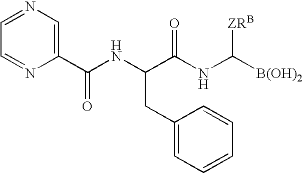
wherein R^{B} is a phosphorus-containing moiety of Series Ia: 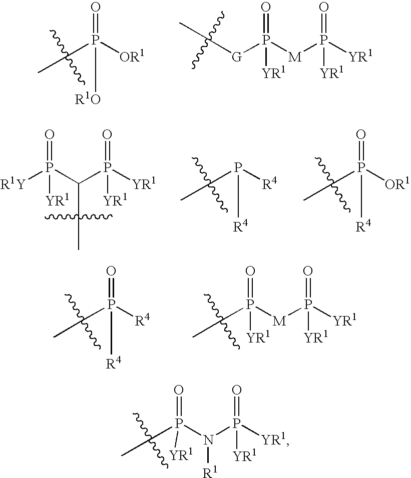
wherein each occurrence of Y is independently —O—, —S—, —NR¹—, or a chemical bond linking R¹ to P,
each occurrence of R¹ is independently a substituted or unsubstituted aliphatic, heteroaliphatic, aryl or heteroaryl moiety, or, except in YR¹ moieties in which Y is a covalent bond, R¹ may also be H;
each occurrence of G is independently absent, or is —O—, —S—, —NR¹— or (M)_{X};
each occurrence of M is independently a substituted or unsubstituted methylene moiety, and any M-M′ moiety may be saturated or unsaturated;
each occurrence of x is independently an integer from 0-6; and
each occurrence of R⁴ is independently alkyl, arylalkyl, aryl or a prodrug moiety.

47. A pharmaceutical composition comprising any one of the compounds of claims 1-7, or 39-46, or a pharmaceutically acceptable derivative thereof; and
a pharmaceutically acceptable carrier or diluent,
said composition optionally further comprising an additional therapeutic agent.

48. The composition of claim 47, wherein the composition further comprises an additional therapeutic agent and the therapeutic agent is an anticancer agent, or an approved agent for the treatment of osteoporosis.

49. A method for treating a bone-related disorder comprising administering a therapeutically effective amount of any one of compounds 1-7, or 39-46, or a pharmaceutically acceptable derivative thereof, to a subject in need thereof,
and optionally further comprising administering an additional therapeutic agent.

50. A method for treating cancer comprising administering a therapeutically effective amount of any one of compounds 1-7, or 39-46, or a pharmaceutically acceptable derivative thereof, to a subject in need thereof,
and optionally further comprising administering an additional therapeutic agent.

51. A method for inhibiting cell adhesion in a mammal comprising administering an effective amount of any one of compounds 1-7, or 39-46, or a pharmaceutically acceptable derivative thereof, to a subject in need thereof.

52. A method for inhibiting HIV infection comprising administering an effective amount of any one of compounds 1-7, or 39-46, or a pharmaceutically acceptable derivative thereof, to a subject in need thereof.

53. A method for inhibiting cyclic degradation in a cell comprising contacting a cell with an effective amount of any one of compounds 1-7, or 39-46, or a pharmaceutically acceptable derivative thereof.

54. A method for treating an inflammatory disorder comprising administering a therapeutically effective amount of any one of compounds 1-7, or 39-46, or a pharmaceutically acceptable derivative thereof, to a subject in need thereof,
and optionally further comprising administering an additional therapeutic agent.

55. A method reducing the activity of NF-kB in a cell comprising contacting a cell with an effective amount of any one of compounds 1-7, or 39-46, or a pharmaceutically acceptable derivative thereof.
